SEQUENCE LISTING (45 sequences)

SEQ ID NO: 1 - DNA, 18 bp, Artificial Sequence
  Description of Artificial Sequence Synthetic primer
gccgatgagttcgacatt

SEQ ID NO: 2 - DNA, 23 bp, Artificial Sequence
  Description of Artificial Sequence Synthetic primer
ttttagaactaagtcttctgctt

SEQ ID NO: 3 - DNA, 46 bp, Artificial Sequence
  Description of Artificial Sequence Synthetic primer
cgcatatattttggccgctagaaaaccacatactttccctatcaca

SEQ ID NO: 4 - DNA, 46 bp, Artificial Sequence
  Description of Artificial Sequence Synthetic primer
taggggatcgattgaaactctttttacaatgctcttgcatattacg

SEQ ID NO: 5 - DNA, 22 bp, Artificial Sequence
  Description of Artificial Sequence Synthetic primer
ggcgatttaaaaaccaaggtcg

SEQ ID NO: 6 - DNA, 20 bp, Artificial Sequence
  Description of Artificial Sequence Synthetic primer
gtagagtttggttggggagg

SEQ ID NO: 7 - DNA, 40 bp, Artificial Sequence
  Description of Artificial Sequence Synthetic primer
caccaagtggtgcaaggatccgacggaattctgtgggaag

SEQ ID NO: 8 - DNA, 47 bp, Artificial Sequence
  Description of Artificial Sequence Synthetic primer
tatcagcatgcgtatgggttactacccatttggaattctttattcac

SEQ ID NO: 9 - DNA, 18 bp, Artificial Sequence
  Description of Artificial Sequence Synthetic primer
ctgctgaaccaagcctta

SEQ ID NO: 10 - DNA, 18 bp, Artificial Sequence
  Description of Artificial Sequence Synthetic primer
gcctgtagcagttgtagg

SEQ ID NO: 11 - DNA, 19 bp, Artificial Sequence
  Description of Artificial Sequence Synthetic primer
aagcggcgtttgtaaacat

SEQ ID NO: 12 - DNA, 18 bp, Artificial Sequence
  Description of Artificial Sequence Synthetic primer
aaggatctccgccgaaac

SEQ ID NO: 13 - DNA, 23 bp, Artificial Sequence
  Description of Artificial Sequence Synthetic primer
gttttcgaccgtgttttgcaaac

SEQ ID NO: 14 - DNA, 20 bp, Artificial Sequence
  Description of Artificial Sequence Synthetic primer
gccggtatggtttcaagacg

SEQ ID NO: 15 - DNA, 20 bp, Artificial Sequence
  Description of Artificial Sequence Synthetic primer
gcccgcaacaaaaagaatcc

SEQ ID NO: 16 - DNA, 19 bp, Artificial Sequence
  Description of Artificial Sequence Synthetic primer
gcagacggagaagcctaac

SEQ ID NO: 17 - DNA, 20 bp, Artificial Sequence
  Description of Artificial Sequence Synthetic primer
tgttgccaaacttggtcagt

SEQ ID NO: 18 - DNA, 38 bp, Artificial Sequence
  Description of Artificial Sequence Synthetic primer
ggctgcggtcattctgcctatgcaaggaaggcgtgaag

SEQ ID NO: 19 - DNA, 39 bp, Artificial Sequence
  Description of Artificial Sequence Synthetic primer
tgcagcgttcgcaggcttaatttccccttacgcttgtcc

SEQ ID NO: 20 - DNA, 20 bp, Artificial Sequence
  Description of Artificial Sequence Synthetic primer
cctgttgccaaacttggtca

SEQ ID NO: 21 - DNA, 21 bp, Artificial Sequence
  Description of Artificial Sequence Synthetic primer
acttcatcaaaaggctggaag

SEQ ID NO: 22 - DNA, 18 bp, Artificial Sequence
  Description of Artificial Sequence Synthetic primer
acgcaataacggcgagtt

SEQ ID NO: 23 - DNA, 40 bp, Artificial Sequence
  Description of Artificial Sequence Synthetic primer
ggcataaacaagacggcgcgtagcgtaaggggaaaaggca

SEQ ID NO: 24 - DNA, 41 bp, Artificial Sequence
  Description of Artificial Sequence Synthetic primer
accggataagggcatttcccgtcaatgctgcgatgatgact

SEQ ID NO: 25 - DNA, 21 bp, Artificial Sequence
  Description of Artificial Sequence Synthetic primer
ggcgatttaaaaaccaaggtc

SEQ ID NO: 26 - DNA, 21 bp, Artificial Sequence
  Description of Artificial Sequence Synthetic primer
tccaatatgattccacccatg

SEQ ID NO: 27 - DNA, 18 bp, Artificial Sequence
  Description of Artificial Sequence Synthetic primer
ggagggatctcgctcctg

SEQ ID NO: 28 - DNA, 18 bp, Artificial Sequence
  Description of Artificial Sequence Synthetic primer
tacccctgccatcaacac

SEQ ID NO: 29 - DNA, 17 bp, Artificial Sequence
  Description of Artificial Sequence Synthetic primer
gcgtgggcattttctgc

SEQ ID NO: 30 - DNA, 21 bp, Artificial Sequence
  Description of Artificial Sequence Synthetic primer
acttccgtggcttcttgctgc

SEQ ID NO: 31 - DNA, 20 bp, Artificial Sequence
  Description of Artificial Sequence Synthetic primer
gcgacaatatcgtctacgtc

SEQ ID NO: 32 - DNA, 18 bp, Artificial Sequence
  Description of Artificial Sequence Synthetic primer
gtgctgcgtgttgtagat

SEQ ID NO: 33 - DNA, 23 bp, Artificial Sequence
  Description of Artificial Sequence Synthetic primer
agcacctgccagtaagtcatcgg

SEQ ID NO: 34 - DNA, 18 bp, Artificial Sequence
  Description of Artificial Sequence Synthetic primer
cacaaactcgccgtctta

SEQ ID NO: 35 - DNA, 18 bp, Artificial Sequence
  Description of Artificial Sequence Synthetic primer
catgcggagaacagaaca

SEQ ID NO: 36 - DNA, 21 bp, Artificial Sequence
  Description of Artificial Sequence Synthetic primer
cctgaagacgcacaacgcctc

SEQ ID NO: 37 - DNA, 18 bp, Artificial Sequence
  Description of Artificial Sequence Synthetic primer
ttggcaaagaattcaccc

SEQ ID NO: 38 - DNA, 20 bp, Artificial Sequence
  Description of Artificial Sequence Synthetic primer
cgagcttagtgatacttgtg

SEQ ID NO: 39 - DNA, 22 bp, Artificial Sequence
  Description of Artificial Sequence Synthetic primer
ttagccacaccagccaccactt

SEQ ID NO: 40 - DNA, 19 bp, Artificial Sequence
  Description of Artificial Sequence Synthetic primer
gcgagtgttgcgagacaac

SEQ ID NO: 41 - DNA, 18 bp, Artificial Sequence
  Description of Artificial Sequence Synthetic primer
caagggacttggacaccg

SEQ ID NO: 42 - DNA, 42 bp, Artificial Sequence
  Description of Artificial Sequence Synthetic primer
actgaggcaatccctgttggttaggtaggaatacccgctgaa

SEQ ID NO: 43 - DNA, 41 bp, Artificial Sequence
  Description of Artificial Sequence Synthetic primer
tgaagcggcaagagctcagatttccacacagactccaacct

SEQ ID NO: 44 - DNA, 24 bp, Artificial Sequence
  Description of Artificial Sequence Synthetic primer
cctccgcttattgatatgcttaag

SEQ ID NO: 45 - DNA, 17 bp, Artificial Sequence
  Description of Artificial Sequence Synthetic primer
gtgctttgcggcacgag

(19) United States
(12) Patent Application Publication
(10) Pub. No.: US 2023/0002805 A1
(43) Pub. Date: Jan. 5, 2023

(54) USE OF ORGANIC CATIONIC COMPOUNDS TO ACCELERATE NUCLEIC ACID HYBRIDIZATION, SYNTHESIS, AND AMPLIFICATION

(71) Applicant: Enzo Biochem, Inc., New York, NY (US)

(72) Inventors: Elazar Rabbani, New York, NY (US); Jack Coleman, East Northport, NY (US)

(73) Assignee: Enzo Biochem, Inc., New York, NY (US)

(21) Appl. No.: 17/551,340

(22) Filed: Dec. 15, 2021

Related U.S. Application Data
(60) Provisional application No. 63/214,862, filed on Jun. 25, 2021.
(60) Provisional application No. 63/216,022, filed on Jun. 29, 2021.
(60) Provisional application No. 63/217,822, filed on Jul. 2, 2021.

(51) Int. Cl. C12Q 1/6806; C12Q 1/6848

(52) U.S. Cl. CPC C12Q 1/6806; C12Q 1/6848

(57) ABSTRACT

The invention provides methods for accelerated synthesis of nucleic acids, and related compositions which involve the use of organic amines in the nucleic acid synthesis reaction mixture. The invention also provides methods for reducing processing steps associated with nucleic acid synthesis. The invention further provides methods for screening compounds that have positive benefits on the synthesis of nucleic acids.

### CROSS-REFERENCE TO RELATED APPLICATION

This application claims the benefit of provisional application No. 63/214,862 filed Jun. 25, 2021, and claims the benefit of provisional application No. 63/216,022 filed Jun. 29, 2021, and claims the benefit of provisional application No. 63/217,822 filed Jul. 2, 2021, all of which are herein incorporated in their entireties.

### FIELD OF THE INVENTION

The invention relates to the field of nucleic acid amplification methods.

### SEQUENCE LISTING STATEMENT

The instant application contains a Sequence Listing which has been submitted electronically in ASCII format and is hereby incorporated by reference in its entirety. Said ASCII copy, created on Dec. 30, 2021, is named ENZ-124-US1-SL.txt and is 10,574 bytes in size.

### BACKGROUND OF THE INVENTION

In the field of nucleic acid hybridization, synthesis, and amplification, sample processing represents some of the most expensive and time consuming steps. A non-exhaustive list of sample processing issues include, for example, the need to remove contaminants from the sample, compounds in the sample that inhibit nucleic acid amplification, the conditions needed to lyse certain pathogens of interest, and/or sample processing conditions that are not compatible with nucleic acid synthesis. There is a need in the art to reduce the amount of sample processing, the time sample processing takes before a sample can undergo amplification and to reduce the cost associated with sample processing.

Improvements in nucleic acid synthesis during nucleic acid amplification is another area of significant research. Much effort is being directed to improving the efficiency and/or speed by which nucleic acids are synthesized during nucleic acid amplification. There is significant interest in methods and compositions that can improve nucleic acid melting temperatures, the annealing process, and/or nucleic acid synthesis during nucleic acid amplification.

Additionally, a process for screening compounds that increase the melting temperature of nucleic acids, improve the annealing process and/or improve nucleic acid synthesis is an active area of research and the object of the present invention.

Isothermal nucleic acid amplification methods are currently being developed for point-of-care (POC) molecular diagnostic clinical testing applications. In this POC environment, rapidity of testing and accuracy is of the essence. Major issues with providing POC testing include: the amount of pre-processing that needs to occur before the test can be performed, the time it takes for results to be obtained, and the ease of obtaining and interpreting the results.

What is needed and provided by the present invention are methods, processes and compositions for accelerating and improving nucleic acid hybridization, synthesis, and amplification.

### SUMMARY OF THE INVENTION

One embodiment of the invention provides a method for nucleic acid hybridization, synthesis, and amplification, which includes providing an amplification reaction buffer solution which may include one or more organic amines in which the amplification is performed. The one or more organic amines may, for example, include: a C₂-C₃₀ amine, a C₂-C₁₂ amine, a C₂-C₃₀ diamine, a C₂-C₁₂ diamine, ethylenediamine; 4,7,10-trioxa-1,13-tridecanediamine; tris(2-aminoethyl)amine; spermidine; 1,3-diaminopropane; putrescine; cadaverine; 1,6-diaminohexane; and/or 2,2′-(Ethylenedioxy)bis(ethylamine). The organic amine may also be, for example, substituted with the following: hydroxyl, —NH—NH₂, halogen, halogen-substituted amino, O-alkyl, alkenyl, alkynyl, nitro, cyano, imino, aminoalkyl, trifluoromethyl, alkoxy, thiocarbonyl, aminoalkyl, sulfonyl, sulfhydryl, unsubstituted cycloalkyl, cycloalkyl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfyhdryl, unsubstituted aryl, aryl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfthydryl. The reaction amplification buffer may also include magnesium ion or magnesium salt, such as MgSO₄, a buffer such as Tris or HEPES, a salt such as sodium chloride or potassium chloride, a detergent such as Tween 20 or Triton X-100, deoxy nucleotides, such as dATP, dCTP, dGTP and TTP or modifications of these, and/or dyes such as phenol red, SyBr green or hydroxynaphthol blue.

Isothermal nucleic acid amplification methods of the present invention include, for example, loop-mediated isothermal amplification (LAMP); nucleic acid sequence-based amplification (NASBA); exponential strand displacement amplification (E-SDA); primer-generation rolling circle amplification (PG-RCA); hyperbranched rolling circle amplification (HRCA); exponential rolling circle amplification (E-RCA); helicase-dependent amplification (HDA); recombinase polymerase amplification (RPA); exponential amplification reaction (EXPAR); whole genome amplification (WGA); transcription mediated amplification (TMA); smart amplification process version 2 (SMAP 2); beacon-assisted detection amplification; linear strand displacement amplification; linear rolling circle amplification; transcription-based amplification; strand displacement-combined cascade amplification; rolling circle-combined cascade amplification.

A related embodiment provides an isothermal nucleic acid amplification reaction mixture or buffer solution, which includes one or more organic amines. The one or more organic amines may, for example, include: a C₂-C₃₀ amine, a C₂-C₁₂ amine, a C₂-C₃₀ diamine, a C₂-C₁₂ diamine, ethylenediamine; 4,7,10-trioxa-1,13-tridecanediamine; tris(2-aminoethyl)amine; spermidine; 1,3-diaminopropane; putrescine; cadaverine; 1,6-diaminohexane; and/or 2,2′-(Ethylenedioxy)bis(ethylamine). The organic amine may also be, for example, substituted with the following: hydroxyl, —NH—NH₂, halogen, halogen-substituted amino, O-alkyl, alkenyl, alkynyl, nitro, cyano, imino, aminoalkyl, trifluoromethyl, alkoxy, thiocarbonyl, aminoalkyl, sulfonyl, sulfhydryl, unsubstituted cycloalkyl, cycloalkyl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfyhdryl, unsubstituted aryl, aryl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfhydryl. The reaction amplification buffer may also include magnesium ion or magnesium salt, such as MgSO₄, a buffer such as Tris or HEPES, a salt such as sodium chloride or potassium chloride, a detergent such as Tween 20 or Triton X-100, deoxy nucleotides, such as dATP, dCTP, dGTP and TTP or modifications of these, dyes such as phenol red, SyBr green or hydroxynaphthol blue.

Another embodiment of the invention provides for a method of reducing background signal in an isothermal nucleic acid amplification, which includes providing an amplification reaction buffer solution which includes one or more organic amines. The one or more organic amines may, for example, include: a C₂-C₃₀ amine, a C₂-C₁₂ amine, a C₂-C₃₀ diamine, a C₂-C₁₂ diamine, ethylenediamine; 4,7,10-trioxa-1,13-tridecanediamine; tris(2-aminoethyl)amine; spermidine; 1,3-diaminopropane; putrescine; cadaverine; 1,6-diaminohexane; and/or 2,2′-(Ethylenedioxy)bis(ethylamine). The organic amine may also be, for example, substituted with the following: hydroxyl, —NH—NH₂, halogen, halogen-substituted amino, O-alkyl, alkenyl, alkynyl, nitro, cyano, imino, aminoalkyl, trifluoromethyl, alkoxy, thiocarbonyl, aminoalkyl, sulfonyl, sulfhydryl, unsubstituted cycloalkyl, cycloalkyl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfyhdryl, unsubstituted aryl, aryl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfhydryl. The reaction amplification buffer may also include magnesium ion or magnesium salt, such as MgSO₄, a buffer such as Tris or HEPES, a salt such as sodium chloride or potassium chloride, a detergent such as Tween 20 or Triton X-100, deoxy nucleotides, such as dATP, dCTP, dGTP and TTP or modifications of these, dyes such as phenol red, SyBr green or hydroxynaphthol blue.

Another embodiment of the invention provides for a method of increasing the melting temperature of nucleic acids during an isothermal nucleic acid amplification, which includes providing an amplification reaction buffer solution which includes one or more organic amines. The one or more organic amines may, for example, include: a C₂-C₃₀ amine, a C₂-C₁₂ amine, a C₂-C₃₀ diamine, a C₂-C₁₂ diamine, ethylenediamine; 4,7,10-trioxa-1,13-tridecanediamine; tris(2-aminoethyl)amine; spermidine; 1,3-diaminopropane; putrescine; cadaverine; 1,6-diaminohexane; and/or 2,2′-(Ethylenedioxy)bis(ethylamine). The organic amine may also be, for example, substituted with the following: hydroxyl, —NH—NH₂, halogen, halogen-substituted amino, O-alkyl, alkenyl, alkynyl, nitro, cyano, imino, aminoalkyl, trifluoromethyl, alkoxy, thiocarbonyl, aminoalkyl, sulfonyl, sulfhydryl, unsubstituted cycloalkyl, cycloalkyl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfyhdryl, unsubstituted aryl, aryl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfhydryl. The reaction amplification buffer may also include magnesium ion or magnesium salt, such as MgSO₄, a buffer such as Tris or HEPES, a salt such as sodium chloride or potassium chloride, a detergent such as Tween 20 or Triton X-100, deoxy nucleotides, such as dATP, dCTP, dGTP and TTP or modifications of these, dyes such as phenol red, SyBr green or hydroxynaphthol blue.

Another embodiment of the invention provides for a method of increasing specific hybridization of nucleic acids during an isothermal nucleic acid amplification, which includes providing an amplification reaction buffer solution which includes one or more organic amines. The one or more organic amines may, for example, include: a C₂-C₃₀ amine, a C₂-C₁₂ amine, a C₂-C₃₀ diamine, a C₂-C₁₂ diamine, ethylenediamine; 4,7,10-trioxa-1,13-tridecanediamine; tris(2-aminoethyl)amine; spermidine; 1,3-diaminopropane; putrescine; cadaverine; 1,6-diaminohexane; and/or 2,2′-(Ethylenedioxy)bis(ethylamine). The organic amine may also be, for example, substituted with the following: hydroxyl, —NH—NH₂, halogen, halogen-substituted amino, O-alkyl, alkenyl, alkynyl, nitro, cyano, imino, aminoalkyl, trifluoromethyl, alkoxy, thiocarbonyl, aminoalkyl, sulfonyl, sulfhydryl, unsubstituted cycloalkyl, cycloalkyl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfyhdryl, unsubstituted aryl, aryl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfhydryl. The reaction amplification buffer may also include magnesium ion or magnesium salt, such as MgSO₄, a buffer such as Tris or HEPES, a salt such as sodium chloride or potassium chloride, a detergent such as Tween 20 or Triton X-100, deoxy nucleotides, such as dATP, dCTP, dGTP and TTP or modifications of these, dyes such as phenol red, SyBr green or hydroxynaphthol blue.

Another embodiment of the invention provides for a method of increasing the melting temperature of nucleic acids and increasing specific hybridization of nucleic acids during an isothermal nucleic acid amplification, which includes providing an amplification reaction buffer solution which includes one or more organic amines. The one or more organic amines may, for example, include: a C₂-C₃₀ amine, a C₂-C₁₂ amine, a C₂-C₃₀ diamine, a C₂-C₁₂ diamine, ethylenediamine; 4,7,10-trioxa-1,13-tridecanediamine; tris(2-aminoethyl)amine; spermidine; 1,3-diaminopropane; putrescine; cadaverine; 1,6-diaminohexane; and/or 2,2′-(Ethylenedioxy)bis(ethylamine). The organic amine may also be, for example, substituted with the following: hydroxyl, —NH—NH₂, halogen, halogen-substituted amino, O-alkyl, alkenyl, alkynyl, nitro, cyano, imino, aminoalkyl, trifluoromethyl, alkoxy, thiocarbonyl, aminoalkyl, sulfonyl, sulfhydryl, unsubstituted cycloalkyl, cycloalkyl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfyhdryl, unsubstituted aryl, aryl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfhydryl. The reaction amplification buffer may also include magnesium ion or magnesium salt, such as MgSO₄, a buffer such as Tris or HEPES, a salt such as sodium chloride or potassium chloride, a detergent such as Tween 20 or Triton X-100, deoxy nucleotides, such as dATP, dCTP, dGTP and TTP or modifications of these, dyes such as phenol red, SyBr green or hydroxynaphthol blue.

Another embodiment of the invention provides for an isothermal amplification sample processing method, which includes adding a sample suspected of containing an analyte to a reaction mixture or buffer solution, which includes one or more organic amines. The one or more organic amines may, for example, include: a C₂-C₃₀ amine, a C₂-C₁₂ amine, a C₂-C₃₀ diamine, a C₂-C₁₂ diamine, ethylenediamine; 4,7,10-trioxa-1,13-tridecanediamine; tris(2-aminoethyl)amine; spermidine; 1,3-diaminopropane; putrescine; cadaverine; 1,6-diaminohexane; and/or 2,2′-(Ethylenedioxy)bis(ethylamine). The organic amine may also be, for example, substituted with the following: hydroxyl, —NH—NH₂, halogen, halogen-substituted amino, O-alkyl, alkenyl, alkynyl, nitro, cyano, imino, aminoalkyl, trifluoromethyl, alkoxy, thiocarbonyl, aminoalkyl, sulfonyl, sulfhydryl, unsubstituted cycloalkyl, cycloalkyl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfyhdryl, unsubstituted aryl, aryl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfhydryl. The reaction amplification buffer may also include magnesium ion or magnesium salt, such as MgSO₄, a buffer such as Tris or HEPES, a salt such as sodium chloride or potassium chloride, a detergent such as Tween 20 or Triton X-100, deoxy nucleotides, such as dATP, dCTP, dGTP and TTP or modifications of these, dyes such as phenol red, SyBr green or hydroxynaphthol blue.

Another embodiment of the invention provides for an amplification sample processing method, which includes adding a sample suspected of containing an analyte to a reaction mixture or buffer solution, which includes urea.

Another embodiment of the invention provides for an amplification sample processing method, which includes adding a sample suspected of containing an analyte to a reaction mixture or buffer solution, which includes urine.

Another embodiment of the invention provides for a process for identifying compounds that can increase the melting temperature of a nucleic acid, increase the specific hybridization of a nucleic acid during an amplification, and improve nucleic acid synthesis, which includes contacting a compound of interest with a nucleic acid having a known melting temperature during amplification and known maximum temperature at which hybridization occurs during amplification and measuring the melting temperature of the nucleic acid after contact with the compound of interest and measuring the maximum temperature at which hybridization occurs after contact with the compound of interest during an isothermal amplification.

Another embodiment of the present invention is platform that reduces or eliminates the necessity to use one or more of the following steps when processing a sample: lysing, purification, and/or extraction. The sample can include, serum, blood, plasma, urine, cerebrospinal fluid, saliva, sputum, or a swab of the nose, mouth, anus, or vagina of an animal, for example, a mammal.

Embodiments of the present invention can also include non-isothermal nucleic acid amplification. For example, the present invention encompasses: Polymerase Chain Reaction (PCR), Real-time PCR, Quantitative real time PCR (Q-RT PCR), Reverse Transcriptase PCR (RT-PCR), Multiplex PCR, Nested PCR, Long-range PCR, Single-cell PCR, Fast-cycling PCR, Methylation-specific PCR (MSP), Hot start PCR, High-fidelity PCR, In situ PCR, Variable Number of Tandem Repeats (VNTR) PCR, Asymmetric PCR, Repetitive sequence-based PCR, Overlap extension PCR, Assemble PCR, Intersequence-specific PCR (ISSR), Ligation-mediated PCR, Methylation-specifin PCR, Miniprimer PCR, Solid phase PCR, and Touch down PCR.

Embodiments of the present invention also include the use of metal ions which confer a benefit in melting temperature, annealing and/or synthesis in isothermal nucleic acid amplification.

Embodiments of the present invention also include the use of organometallic compounds which confer a benefit in melting temperature, annealing and/or synthesis in isothermal nucleic acid amplification.

Embodiments of the present invention also include the use of other cations which confer a benefit in melting temperature, annealing and/or synthesis in isothermal nucleic acid amplification.

Additional features, advantages, and embodiments of the invention may be set forth or apparent from consideration of the following detailed description, drawings if any, and claims.

Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1 shows Table 4—effect of various polyamines on loop-mediated isothermal amplification of nucleic acids.

FIG. 2 shows Table 5—effect of putrescine on PCR reactions.

FIG. 3 shows Table 11—effect of various additives on loop-mediated isothermal amplification of nucleic acids with 2.5 mM MgSO₄ concentration.

FIG. 4 shows the use of putrescine significantly reduces background signals in qPCR reactions.

FIGS. 5A to 5D show the addition of putrescine enhances qPCR sensitivity.

FIG. 6 shows the effects of different organic amines on isothermal amplification.

FIG. 7 shows the titration of Neisseria or Chlamydia target with or without putrescine.

FIG. 8 shows Ct values with and without 16 mM putrescine in Women's Health (AmpiProbe) amplification.

FIG. 9 shows a serial dilution with putrescine added to Enzo Universal RT Mix.

FIG. 10 shows a PCR reaction of Seracare Neisseria/Chlamydia with urine, with and without heating to 95° C.

FIG. 11 shows Ct measurements for a selection of diamines.

FIG. 12 shows an assay using the ENZO Universal PCR Taq, with or without an additional 4 mM putrescine.

FIG. 13 shows methods to release DNA from Candida krusei.

### DETAILED DESCRIPTION

As used herein, organic amines means a carbon based compound that contains at least one amine group attached to a carbon atom. Preferably, the organic amine contains 2 to 30 carbon atoms. More preferably the organic amine contains 2 to 12 carbon atoms. The organic amine can be aliphatic. The organic amine can be linear, branched, aromatic and/or substituted at one or more positions. The organic amine can be a diamine. The organic amine can be an alkyl diamine. Preferably, the organic amine can be a linear alkyl diamine, more preferably a linear alkyl diamine wherein an amino group is disposed at each end. Organic amines of the present invention include, for example: a C₂-C₃₀ amine, a C₂-C₁₂ amine, a C₂-C₃₀ diamine, a C₂-C₁₂ diamine, ethylenediamine; 4,7,10-trioxa-1,13-tridecanediamine; tris(2-aminoethyl)amine; spermidine; 1,3-diaminopropane; putrescine; cadaverine; 1,6-diaminohexane; and/or 2,2′-(Ethylenedioxy)bis(ethylamine). The organic amine may also be, for example, substituted with the following: hydroxyl, —NH—NH₂, halogen, halogen-substituted amino, O-alkyl, alkenyl, alkynyl, nitro, cyano, imino, aminoalkyl, trifluoromethyl, alkoxy, thiocarbonyl, aminoalkyl, sulfonyl, sulfhydryl, unsubstituted cycloalkyl, cycloalkyl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfyhdryl, unsubstituted aryl, aryl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfhydryl.

Isothermal amplification methods of the present invention include, for example, loop-mediated isothermal amplification (LAMP); nucleic acid sequence-based amplification (NASBA); exponential strand displacement amplification (E-SDA); primer-generation rolling circle amplification (PG-RCA); hyperbranched rolling circle amplification (HRCA); exponential rolling circle amplification (E-RCA); helicase-dependent amplification (HDA); recombinase polymerase amplification (RPA); exponential amplification reaction (EXPAR); whole genome amplification (WGA); transcription mediated amplification (TMA); smart amplification process version 2 (SMAP 2); beacon-assisted detection amplification; linear strand displacement amplification; linear rolling circle amplification; transcription-based amplification; strand displacement-combined cascade amplification; rolling circle-combined cascade amplification.

Non-isothermal amplification methods of the present invention include, for example, Real-time PCR, Quantitative real time PCR (Q-RT PCR), Reverse Transcriptase PCR (RT-PCR), Multiplex PCR, Nested PCR, Long-range PCR, Single-cell PCR, Fast-cycling PCR, Methylation-specific PCR (MSP), Hot start PCR, High-fidelity PCR, In situ PCR, Variable Number of Tandem Repeats (VNTR) PCR, Asymmetric PCR, Repetitive sequence-based PCR, Overlap extension PCR, Assemble PCR, Intersequence-specific PCR (ISSR), Ligation-mediated PCR, Methylation-specifin PCR, Miniprimer PCR, Solid phase PCR, and Touch down PCR.

Accelerating Nucleic Acid Amplification with Cations

The invention provides methods for enhanced synthesis, hybridization, and/or amplification of nucleic acids, and related compositions which involve the use of organic amines in the amplification reaction mixture. The organic amines may, for example, include: a C₂-C₃₀ amine, a C₂-C₁₂ amine, a C₂-C₃₀ diamine, a C₂-C₁₂ diamine, ethylenediamine; 4,7,10-trioxa-1,13-tridecanediamine; tris(2-aminoethyl)amine; spermidine; 1,3-diaminopropane; putrescine; cadaverine; 1,6-diaminohexane; and/or 2,2′-(Ethylenedioxy)bis(ethylamine). The organic amine may also be, for example, substituted with the following: hydroxyl, —NH—NH₂, halogen, halogen-substituted amino, O-alkyl, alkenyl, alkynyl, nitro, cyano, imino, aminoalkyl, trifluoromethyl, alkoxy, thiocarbonyl, aminoalkyl, sulfonyl, sulfhydryl, unsubstituted cycloalkyl, cycloalkyl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfyhdryl, unsubstituted aryl, aryl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfhydryl. The reaction amplification buffer may also include magnesium ion or magnesium salt, such as MgSO₄, a buffer such as Tris or HEPES, a salt such as sodium chloride or potassium chloride, a detergent such as Tween 20 or Triton X-100, deoxy nucleotides, such as dATP, dCTP, dGTP and TTP or modifications of these, dyes such as phenol red, SyBr green or hydroxynaphthol blue.

The present invention also includes the use of one or more organic amines which confer a benefit in melting temperature, annealing and/or synthesis in nucleic acid amplification, including, but not limited to isothermal nucleic acid amplification. For example, the one or more organic amines may increase the melting temperature of nucleic acids. The one or more organic amines may increase the efficiency and specificity or the annealing stage of isothermal nucleic acid amplification. The one or more organic amines may speed up the synthesis step of isothermal nucleic acid amplification, resulting in faster results.

The invention also provides for nucleic acid amplification reaction mixtures or buffers, which includes one or more organic amines. The one or more organic amines may, for example, include: a C₂-C₃₀ amine, a C₂-C₁₂ amine, a C₂-C₃₀ diamine, a C₂-C₁₂ diamine, ethylenediamine; 4,7,10-trioxa-1,13-tridecanediamine; tris(2-aminoethyl)amine; spermidine; 1,3-diaminopropane; putrescine; cadaverine; 1,6-diaminohexane; and/or 2,2′-(Ethylenedioxy)bis(ethylamine). The organic amine may also be, for example, substituted with the following: hydroxyl, —NH—NH₂, halogen, halogen-substituted amino, O-alkyl, alkenyl, alkynyl, nitro, cyano, imino, aminoalkyl, trifluoromethyl, alkoxy, thiocarbonyl, aminoalkyl, sulfonyl, sulfhydryl, unsubstituted cycloalkyl, cycloalkyl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfyhdryl, unsubstituted aryl, aryl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfhydryl. The reaction amplification buffer may also include magnesium ion or magnesium salt, such as MgSO₄, a buffer such as Tris or HEPES, a salt such as sodium chloride or potassium chloride, a detergent such as Tween 20 or Triton X-100, deoxy nucleotides, such as dATP, dCTP, dGTP and TTP or modifications of these, dyes such as phenol red, SyBr green or hydroxynaphthol blue.

The invention also provides for a method of reducing background signal in a nucleic acid amplification, which includes providing an amplification reaction buffer solution which includes one or more organic amines. The one or more organic amines may, for example, include: a C₂-C₃₀ amine, a C₂-C₁₂ amine, a C₂-C₃₀ diamine, a C₂-C₁₂ diamine, ethylenediamine; 4,7,10-trioxa-1,13-tridecanediamine; tris(2-aminoethyl)amine; spermidine; 1,3-diaminopropane; putrescine; cadaverine; 1,6-diaminohexane; and/or 2,2′-(Ethylenedioxy)bis(ethylamine). The organic amine may also be, for example, substituted with the following: hydroxyl, —NH—NH₂, halogen, halogen-substituted amino, O-alkyl, alkenyl, alkynyl, nitro, cyano, imino, aminoalkyl, trifluoromethyl, alkoxy, thiocarbonyl, aminoalkyl, sulfonyl, sulfhydryl, unsubstituted cycloalkyl, cycloalkyl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfyhdryl, unsubstituted aryl, aryl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfhydryl. The reaction amplification buffer may also include magnesium ion or magnesium salt, such as MgSO₄, a buffer such as Tris or HEPES, a salt such as sodium chloride or potassium chloride, a detergent such as Tween 20 or Triton X-100, deoxy nucleotides, such as dATP, dCTP, dGTP and TTP or modifications of these, dyes such as phenol red, SyBr green or hydroxynaphthol blue.

The invention also provides for a method of increasing the melting temperature of nucleic acids during a nucleic acid amplification, which includes providing an amplification reaction buffer solution which includes one or more organic amines. The one or more organic amines may, for example, include: a C₂-C₃₀ amine, a C₂-C₁₂ amine, a C₂-C₃₀ diamine, a C₂-C₁₂ diamine, ethylenediamine; 4,7,10-trioxa-1,13-tridecanediamine; tris(2-aminoethyl)amine; spermidine; 1,3-diaminopropane; putrescine; cadaverine; 1,6-diaminohexane; and/or 2,2′-(Ethylenedioxy)bis(ethylamine). The organic amine may also be, for example, substituted with the following: hydroxyl, —NH—NH₂, halogen, halogen-substituted amino, O-alkyl, alkenyl, alkynyl, nitro, cyano, imino, aminoalkyl, trifluoromethyl, alkoxy, thiocarbonyl, aminoalkyl, sulfonyl, sulfhydryl, unsubstituted cycloalkyl, cycloalkyl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfyhdryl, unsubstituted aryl, aryl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfhydryl. The reaction amplification buffer may also include magnesium ion or magnesium salt, such as MgSO₄, a buffer such as Tris or HEPES, a salt such as sodium chloride or potassium chloride, a detergent such as Tween 20 or Triton X-100, deoxy nucleotides, such as dATP, dCTP, dGTP and TTP or modifications of these, dyes such as phenol red, SyBr green or hydroxynaphthol blue.

The invention also provides for a method of increasing specific hybridization of a nucleic acids during a nucleic acid amplification, which includes providing an amplification reaction buffer solution which includes one or more organic amines. The one or more organic amines may, for example, include: a C₂-C₃₀ amine, a C₂-C₁₂ amine, a C₂-C₃₀ diamine, a C₂-C₁₂ diamine, ethylenediamine; 4,7,10-trioxa-1,13-tridecanediamine; tris(2-aminoethyl)amine; spermidine; 1,3-diaminopropane; putrescine; cadaverine; 1,6-diaminohexane; and/or 2,2′-(Ethylenedioxy)bis(ethylamine). The organic amine may also be, for example, substituted with the following: hydroxyl, —NH—NH₂, halogen, halogen-substituted amino, O-alkyl, alkenyl, alkynyl, nitro, cyano, imino, aminoalkyl, trifluoromethyl, alkoxy, thiocarbonyl, aminoalkyl, sulfonyl, sulfhydryl, unsubstituted cycloalkyl, cycloalkyl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfyhdryl, unsubstituted aryl, aryl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfhydryl. The reaction amplification buffer may also include magnesium ion or magnesium salt, such as MgSO₄, a buffer such as Tris or HEPES, a salt such as sodium chloride or potassium chloride, a detergent such as Tween 20 or Triton X-100, deoxy nucleotides, such as dATP, dCTP, dGTP and TTP or modifications of these, dyes such as phenol red, SyBr green or hydroxynaphthol blue.

The invention also provides for a method of increasing the melting temperature of nucleic acids and increasing specific hybridization of nucleic acids during a nucleic acid amplification, which includes providing an amplification reaction buffer solution which includes one or more organic amines. The one or more organic amines may, for example, include: a C₂-C₃₀ amine, a C₂-C₁₂ amine, a C₂-C₃₀ diamine, a C₂-C₁₂ diamine, ethylenediamine; 4,7,10-trioxa-1,13-tridecanediamine; tris(2-aminoethyl)amine; spermidine; 1,3-diaminopropane; putrescine; cadaverine; 1,6-diaminohexane; and/or 2,2′-(Ethylenedioxy)bis(ethylamine). The organic amine may also be, for example, substituted with the following: hydroxyl, —NH—NH₂, halogen, halogen-substituted amino, O-alkyl, alkenyl, alkynyl, nitro, cyano, imino, aminoalkyl, trifluoromethyl, alkoxy, thiocarbonyl, aminoalkyl, sulfonyl, sulfhydryl, unsubstituted cycloalkyl, cycloalkyl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfyhdryl, unsubstituted aryl, aryl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfhydryl. The reaction amplification buffer may also include magnesium ion or magnesium salt, such as MgSO₄, a buffer such as Tris or HEPES, a salt such as sodium chloride or potassium chloride, a detergent such as Tween 20 or Triton X-100, deoxy nucleotides, such as dATP, dCTP, dGTP and TTP or modifications of these, dyes such as phenol red, SyBr green or hydroxynaphthol blue.

The invention further provides for an isothermal synthesis, hybridization, and amplification sample processing method, which includes adding a sample suspected of containing an analyte to a reaction mixture or buffer solution, which includes one or more organic amines. The one or more organic amines may, for example, include: a C₂-C₃₀ amine, a C₂-C₁₂ amine, a C₂-C₃₀ diamine, a C₂-C₁₂ diamine, ethylenediamine; 4,7,10-trioxa-1,13-tridecanediamine; tris(2-aminoethyl)amine; spermidine; 1,3-diaminopropane; putrescine; cadaverine; 1,6-diaminohexane; and/or 2,2′-(Ethylenedioxy)bis(ethylamine). The organic amine may also be, for example, substituted with the following: hydroxyl, —NH—NH₂, halogen, halogen-substituted amino, O-alkyl, alkenyl, alkynyl, nitro, cyano, imino, aminoalkyl, trifluoromethyl, alkoxy, thiocarbonyl, aminoalkyl, sulfonyl, sulfhydryl, unsubstituted cycloalkyl, cycloalkyl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfyhdryl, unsubstituted aryl, aryl substituted by halo, haloalkyl, hydroxyl, nitro, trifluoromethyl, C₁-C₆ straight or branched chain alkyl, C₁-C₅ alkoxy, amino, thiocarbonyl, cyano, imino, aminoalkyl, sulfonyl, or sulfhydryl. The reaction amplification buffer may also include magnesium ion or magnesium salt, such as MgSO₄, a buffer such as Tris or HEPES, a salt such as sodium chloride or potassium chloride, a detergent such as Tween 20 or Triton X-100, deoxy nucleotides, such as dATP, dCTP, dGTP and TTP or modifications of these, dyes such as phenol red, SyBr green or hydroxynaphthol blue.

The concentration of organic amine in the present invention can be, for example, between 0.5 and 50 mM in the final concentration of the reaction mixture. Preferably, the organic amine concentration can be, for example, between about 1 mM and about 10 mM, about 2 mM and about 8 mM, or about 4 mM+/−20% of the final concentration of the reaction mixture. The organic amine concentration can be, for example about 0.5 mM, about 1, mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM or about 50 mM of the final concentration of the reaction mixture.

The isothermal reactions of the present invention are performed at a temperature of about 50° C. to about 105° C., preferably about 55° C. to about 90° C., preferably about 55° C. to about 75° C., preferably from about 62° C. to about 68° C. The isothermal reactions of the present invention can be performed at a temperature, for example, of about 50° C., 55° C., 60° C., 62° C., 65° C., 67° C., 68° C., 70° C., 75° C., 80° C., 85° C., 90° C., 95° C., 100° C., 105° C. or any temperature in-between. It is understood that the temperature may fluctuate+/−3° C. during the reaction.

The analytes for this invention can be organic amines. Preferred analytes include ethylenediamine; 4,7,10-trioxa-1,13-tridecanediamine; tris(2-aminoethyl)amine; spermidine; 1,3-diaminopropane; putrescine; cadaverine; 1,6-diaminohexane; and/or 2,2′-(Ethylenedioxy)bis(ethylamine). More preferred analytes include cadaverine, putrescine and/or 1,6-diaminohexane.

As used herein, the term oligoamine includes metal ions, chelators, and organometallic compounds, or combinations thereof.

In another embodiment, the present invention also provides the use of metal ions which confer a benefit in melting temperature, annealing and/or synthesis in nucleic acid amplification. The metals can be used in conjunction with an appropriate chelator. The metal can be added before the chelator, after the chelator, or in combination with the chelator. The chelator can be attached to another chelator through a linker as shown in Scheme 1.

Metal ions that can be used in the present invention include, but are not limited to: alkali and alkaline earth metal ions, such as potassium, lithium, calcium, sodium, rubidium, cesium, strontium, magnesium, and barium; aluminum; mercury; antimony; indium; tin; actinium; transition metals, such as cobalt, titanium, vanadium, gold, ruthenium, rhenium, zirconium, yttrium, scandium, nickel, manganese, zinc, chromium, silver, cadmium, lead, palladium, platinum, iron, and copper; lanthanides, such as lanthanide, cerium, terbium, praseodymium, europium, gadolinium, terbium, lutetium, samarium, and dysprosium, including combinations thereof.

Chelators that can be used in the present invention include, but are not limited to: 1,4,7-triazacyclononane-1,4,7-triacetic acid (NOTA), 1,4,7,10-tetraazacyclododecane-1,4,7,10-tetraacetic acid (DOTA), as well as modified DOTA, BIS-DOTA, and DOTA-amine, 1,2-bis(o-aminophenoxy)ethane-N,N,N′,N′-tetraacetic acid (BAPTA), egtazic acid (EGTA), ethylenediaminetetraacetic acid (EDTA), 2-[6-[Bis(carboxylatomethyl)amino]-5-[2-[2-[bis(carboxylatomethyl)amino]-5-methylphenoxy]ethoxy]-1-benzofuran-2-yl]-1,3-oxazole-5-carboxylate pentapotassium salt (FURA 2), N,N,N′,N′-Tetrakis(2-pyridylmethyl)ethylenediamine (TPEN), Quinoline-Based chelators (such as those disclosed in Fahrni, et al., JACS, 1999, 121:11448-11458, which is incorporated by reference), as well as other chelators known in the art, including combinations and derivatives thereof.

Organometallic compounds that can be used in the present invention include, but are not limited to: ferrocene, cobaltacene, tris(triphenylphosphine)rhodium carbonyl hydride, Zeise's salt, trimethylaluminum, dimethylzinc, Gilman reagents, Grinard reagents, tetracarbonyl nickel, tributyltin hydride, diethylzinc, methylcobalamin, triethylborane, lithium diphenylcuprate bis(diethyl etherate), adenosylcobalamin, iron pentacrabonyl, and technetium sestamibi, including combinations and derivatives thereof.

The metal ion concentration of the present invention can be, for example, between 0.5 and 50 mM in the final concentration of the reaction mixture. Preferably, the metal ion concentration can be, for example, between about 1 mM and about 10 mM, about 2 mM and about 8 mM, or about 4 mM+/−20% of the final concentration of the reaction mixture. The metal ion concentration can be, for example about 0.5 mM, about 1, mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM or about 50 mM of the final concentration of the reaction mixture.

The concentration of chelator in the present invention can be, for example, between 0.5 and 50 mM in the final concentration of the reaction mixture. Preferably, the chelator concentration can be, for example, between about 1 mM and about 10 mM, about 2 mM and about 8 mM, or about 4 mM+/−20% of the final concentration of the reaction mixture. The chelator concentration can be, for example about 0.5 mM, about 1, mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM or about 50 mM of the final concentration of the reaction mixture.

In another embodiment, the present invention also provides the use of organometallic compounds which confer a benefit in melting temperature, annealing and/or synthesis in nucleic acid amplification.

The concentration of organometallic compounds in the present invention can be, for example, between 0.5 and 50 mM in the final concentration of the reaction mixture. Preferably, the organometallic compounds concentration can be, for example, between about 1 mM and about 10 mM, about 2 mM and about 8 mM, or about 4 mM+/−20% of the final concentration of the reaction mixture. The organometallic compounds concentration can be, for example about 0.5 mM, about 1, mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM or about 50 mM of the final concentration of the reaction mixture.

In another embodiment, the present invention also provides the use of other dications or oligocations which confer a benefit in melting temperature, annealing and/or synthesis in nucleic acid amplification. Other cations that can be used in the present invention include, but are not limited to: allyl cations, cycloalkyl cations, tropylium, carbenium, carboxonium, oxycarbenium, and oxonium, including combinations and derivatives thereof.

The concentration of other cations in the present invention can be, for example, between 0.5 and 50 mM in the final concentration of the reaction mixture. Preferably, other cations concentration can be, for example, between about 1 mM and about 10 mM, about 2 mM and about 8 mM, or about 4 mM+/−20% of the final concentration of the reaction mixture. The other cations concentration can be, for example about 0.5 mM, about 1, mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM or about 50 mM of the final concentration of the reaction mixture.

### Screening Compounds for Benefits in Nucleic Acid Synthesis

It has been surprisingly found that compounds that increase both the melting temperature and annealing temperature during nucleic acid synthesis, improve the overall performance of the nucleic acid synthesis. These overall performance improvements includes, for example, a decrease in the time it takes to complete the amplification, resulting in more rapid detection of a target; a more efficient nucleic acid synthesis; a reduction in background noise during the nucleic acid synthesis; and/or a decrease in the cost of the nucleic acid synthesis.

The invention further provides for a process for identifying compounds that can increase the melting temperature of a nucleic acid and/or enhance the hybridization of a nucleic acid during a nucleic acid synthesis, which includes contacting a compound of interest with a nucleic acid having a known melting temperature during synthesis and known maximum temperature at which hybridization occurs during synthesis and measuring the melting temperature of the nucleic acid after contact with the compound of interest and measuring the maximum temperature at which hybridization occurs after contact with the compound of interest during a nucleic acid synthesis. These methods may be used to identify compounds of the present invention that improve nucleic acid synthesis.

### Benefits in Processing of Samples

The present invention provides for a reduction in cost of nucleic acid detection, a reduction of time to perform a nucleic acid detection, and/or an increase in the efficiency of nucleic acid detection.

For the present disclosure, lyse or lysing means disrupting the exterior environment of a cell in a way that causes it to break open and release its contents. For the present disclosure, isolation means binding, precipitation, or separation of nucleic acids. For the present disclosure, elution means eluting in a buffer that is compatible with the amplification system chosen.

Urine and urea are normally inhibitory to nucleic acid detection. It has been surprisingly found that urine and urea can be used to reduce processing steps in nucleic acid detections.

The present invention include the addition of urea or urine to a sample before heating can reduce the time, cost and/or number of processing steps associated with analyzing a sample for specific nucleic acids. The addition of urea or urine allows for some reduction or elimination of processing steps. For example, addition of urea or urine could reduce or eliminate the need to lyse cells in a sample, purify a sample, and/or concentrate a sample before performing nucleic acid detection. This saves not only time but also costs associated with analyzing a sample for specific nucleic acids.

The amount of urea used in the present invention can be, for example, an amount where the concentration of urea in the reaction is between about 1 mM and about 8 M, preferably between about 2 mM and about 4 M, or about 1 mM, about 2 mM, about 3 mM, about 4 mM, about 5 mM, about 6 mM, about 7 mM, about 8 mM, about 9 mM, about 10 mM, about 20 mM, about 30 mM, about 40 mM, about 50 mM, about 75 mM, about 100 mM, about 150 mM, about 200 mM, about 250 mM, about 300 mM, about 400 mM, about 500 mM, about 600 mM, about 700 mM, about 800 mM, about 900 mM, about 1 M, about 1.5 M, about 2 M, about 2.5 M, about 3 M, about 4 M, about 5 M, about 6 M, about 7 M, or about 8 M.

For the purposes of the present invention, urea can also be added to a blood sample in an amount, for example, where the concentration of urea in the blood before heating is between about 200 mM and about 8 M, between about 1 M and 4 M, about 200 mM, about 500 mM, about 750 mM, about 1 M, about 2 M, about 3 M, about 4 M, about 5 M, about 6 M, about 7 M, or about 8 M.

The concentration of urine in the present invention can be, for example, between about 10% to about 50%, about 10%, about 15%, about 20%, about 25%, about 30%, about 35%, about 40%, about 45%, or about 50% of the reaction.

The concentration of magnesium in the present invention can be from about 1 mM to about 10 mM or any concentration value therein, such as about 1 mM, about 2 mM, about 3 mM, about 4 mM, about 5 mM, about 6 mM, about 7 mM, about 8 mM, about 9 mM, about 10 mM, or about 4-10 mM, such as about 2 mM+/−20% mM, such as about 2 mM, such as about 4 mM +/−20% mM, or about 8+/−20% mM.

The invention also provides a platform that reduces or eliminates the necessity to use one or more of the following steps when processing a sample: lysing, isolation, elution, purification, and/or extraction. The sample can include, serum, blood, plasma, urine, sputum, cerebrospinal fluid, saliva or a swab of the nose, mouth, anus, or vagina of an animal, for example, a mammal. Preferably, the sample is from a human. It has been surprisingly found that using the methods and compounds of the present invention eliminate or reduce the need to purify, and/or perform extraction on a sample of interest to be analyzed by an isothermal nucleic acid detection, PCR, Real-time PCR, Quantitative real time PCR (Q-RT PCR), Reverse Transcriptase PCR (RT-PCR), Multiplex PCR, Nested PCR, Long-range PCR, Single-cell PCR, Fast-cycling PCR, Methylation-specific PCR (MSP), Hot start PCR, High-fidelity PCR, In situ PCR, Variable Number of Tandem Repeats (VNTR) PCR, Asymmetric PCR, Repetitive sequence-based PCR, Overlap extension PCR, Assemble PCR, Intersequence-specific PCR (ISSR), Ligation-mediated PCR, Methylation-specifin PCR, Miniprimer PCR, Solid phase PCR, and Touch down PCR.

The methods and compositions of the present invention have numerous benefits, including reducing the complexity, cost, time, and/or number of potential errors that may occur while analyzing a sample. The present invention provides methods to decrease the time to complete isothermal amplifications to allow a more rapid detection of a target. The present invention also provides a method to more efficiently use a sample of blood, cerebrospinal fluid, plasma, saliva or serum directly in the nucleic acid amplification reaction without prior purification of nucleic acids present in the sample. The present invention provides for a decrease in cost for POC nucleic acid amplification and detection. The present invention also provides for a decrease in the amount of sample preparation in a nucleic acid synthesis. The present invention further provides for a decrease in time to obtain results for POC nucleic acid amplification and detection. The present invention also provides for a method of screen compounds that can improve nucleic acid amplification.

### EXAMPLES

### Example 1

Experiments were performed to determine the impact of polyamines and MgSO₄ concentrations on the performance of loop-mediated isothermal amplification (LAMP) test reactions.

The default reaction mixture/buffer composition used (prior to the addition of the additives being tested) is shown in Table 1 below:

TABLE 1
20 mM TrisHCl, pH 8.8
10 mM KCl
10 mM Ammonium sulfate
0.1% Tween-20
960 mM Betaine
0.4 mM dATP
0.4 mM dCTP
0.4 mM dGTP
0.4 mM dTTP
0.5X CyGreen ® Nucleic Acid Dye (Catalog No. ENZ-GEN105-0100;
Enzo Life Sciences, Inc., Farmingdale, NY, USA)
40 mM Guanidine HCl
8 units Bst 2.0 WarmStart ® polymerase (Catalog No. M0538S;
New England Biolabs, Inc., Ipswich, MA, USA)

The reaction was performed at 67° C. The target was Seracare Chlamydia trichomonas/Neisseria gonorrhoeae controls heated to 95° C. for 3 minutes. The primer set used for Seracare Chlamydia trichomonas is shown in Table 2 below:

TABLE 2

| 0.2 μM | CTPlasLF372 | gccgatgagttcgacatt (SEQ ID NO: 1) |
|---|---|---|
| 0.2 μM | CTPlasLR544 | ttttagaactaagtcttctgctt (SEQ ID NO: 2) |
| 1.6 μM | CTPlasFL390 | cgcatatattttggccgctagaaaaccacatactttccctatcaca (SEQ ID |
| | | NO: 3) |
| 1.6 μM | CTPlasRL543 | taggggatcgattgaaactctttttacaatgctcttgcatattacg (SEQ ID |
| | | NO: 4) |
| 0.4 μM | CTPlasLLF412 | ggcgatttaaaaaccaaggtcg (SEQ ID NO: 5) |
| 0.4 μM | CTPlasLLR490 | gtagagtttggttggggagg (SEQ ID NO: 6) |
| 1.6 μM | CtOmp105FLg | caccaagtggtgcaaggatccgacggaattctgtgggaag (SEQ ID |
| | | NO: 7) |
| 1.6 μM | CtOmp254RL | tatcagcatgcgtatgggttactacccatttggaattctttattcac (SEQ ID |
| | | NO: 8) |
| 0.2 μM | CtOmp83FP | ctgctgaaccaagectta (SEQ ID NO: 9) |
| 0.2 μM | CtOmp279RP | gcctgtagcagttgtagg (SEQ ID NO: 10) |
| 0.1 μM | CtOmp370RP | aagcggcgtttgtaaacat (SEQ ID NO: 11) |
| 0.4 μM | CtOmp125LoopF | aaggatctccgccgaaac (SEQ ID NO: 12) |
| 0.4 μM | CtOmp226LoopR | gttttcgaccgtgttttgcaaac (SEQ ID NO: 13) |

The primer set used for Neisseria gonorrhoeae is shown in Table 3 below:

TABLE 3

| | | |
|---|---|---|
| 0.4 μM | Ng-LF1 | GCCGGTATGGTTTCAAGACG (SEQ ID |
| | | NO: 14) |
| 0.4 μM | Ng-LR1 | GCCCGCAACAAAAAGAATCC (SEQ ID |
| | | NO: 15) |
| 0.2 μM | Ng-F1 | GCAGACGGAGAAGCCTAAC (SEQ ID |
| | | NO: 16) |
| 0.2 μM | Ng-R1 | TGTTGCCAAACTTGGTCAGT (SEQ ID |
| | | NO: 17) |
| 1.6 μM | Ng-LPF1 | GGCTGCGGTCATTCTGCCTA |
| | | TGCAAGGAAGGCGTGAAG (SEQ ID |
| | | NO: 18) |
| 1.6 μM | Ng-LPR1 | TGCAGCGTTCGCAGGCTTAA |
| | | TTTCCCCTTACGCTTGTCC (SEQ ID |
| | | NO: 19) |
| 0.4 μM | Ng-LF4 | CCTGTTGCCAAACTTGGTCA (SEQ ID |
| | | NO: 20) |
| 0.4 μM | Ng-LR4 | ACTTCATCAAAAGGCTGGAAG (SEQ ID |
| | | NO: 21) |
| 0.2 μM | Ng-F4 | GCCCGCAACAAAAAGAATCC (SEQ ID |
| | | NO: 15) |
| 0.2 μM | Ng-R4 | ACGCAATAACGGCGAGTT (SEQ ID |
| | | NO: 22) |
| 1.6 μM | N-LPF4 | GGCATAAACAAGACGGCGCGTA |
| | | GCGTAAGGGGAAAAGGCA (SEQ ID |
| | | NO: 23) |
| 1.6 μM | Ng-LPR4 | ACCGGATAAGGGCATTTCCCG |
| | | TCAATGCTGCGATGATGACT (SEQ ID |
| | | NO: 24) |

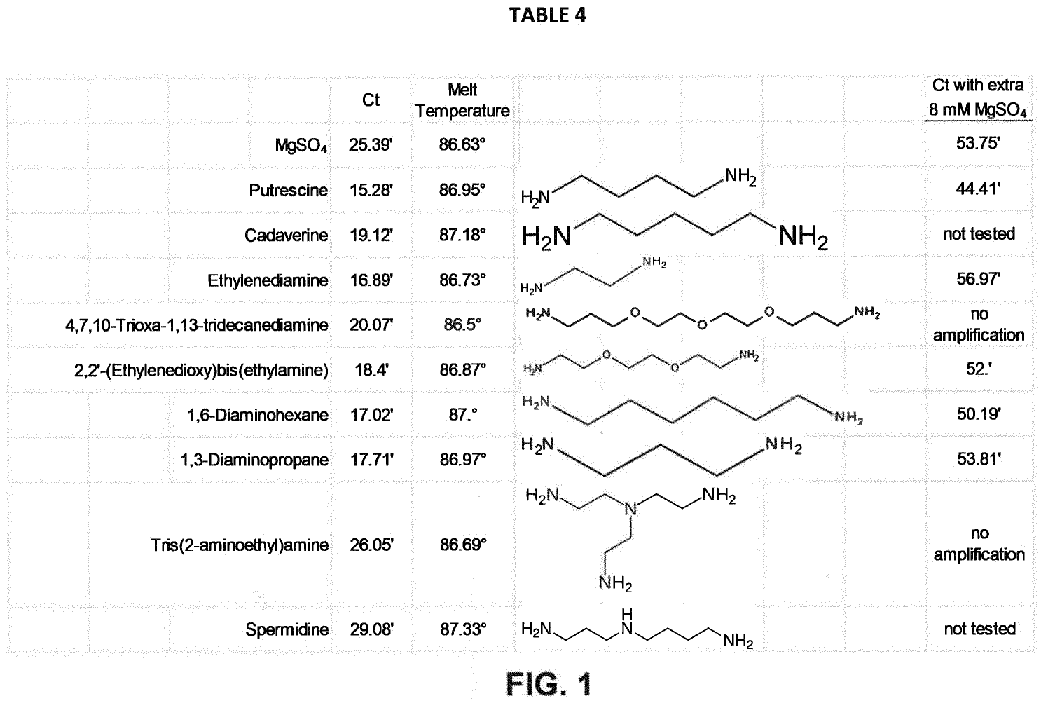

The reactions were constituted with the concentrations of MgSO₄ and/or polyamines and the resulting Ct values (time in minutes to reach threshold value of detection) were determined as shown in Table 4 (appended as FIG. 1). In all cases, there is 4 mM MgSO₄ and 4 mM additive (either more MgSO₄ or a polyamine). Thus, the normal (reference) reaction (indicated as 4 mM MgSO₄) has a total MgSO₄ concentration of 8 mM. The rightmost column shows the results for corresponding reactions in which all reactions had 8 mM MgSO₄.

As shown in Table 4 (FIG. 1), several polyamines very significantly reduced Ct (measured as the time in minutes to reach the threshold of detection for amplification of the target). Among the polyamines tested, putrescine was most effective in reducing Ct.

It should be understood that a basic LAMP reaction mixture in the various LAMP embodiments of the invention may include a buffer solution, a forward internal primer, a forward external primer, a backward internal primer, a backward external primer, optionally a forward loop primer, optionally a backward loop primer, nucleoside triphosphates (the dNTPs—dATP, dTTP, dCTP and dGTP), and a strand-displacing DNA-dependent DNA polymerase, as known in the art. The reaction mixture may include a test sample that may include a nucleic acid target, such as an RNA or DNA target, for amplification. The reaction mixture may also include a compound or component for the detection of amplification/DNA synthesis such as CyGreen®.

### Example 2

To further investigate the effects of the addition of various polyamines in isothermal nucleic amplification reactions as described in the previous example, melting point experiments were performed using 100 ng DNA of a linearized 3 kb plasmid in 20 mM Tris-HCl, pH 8, 50 m KCl, CyGreen diluted 20.000× from stock, with the following components added:

| | | |
|---|---|---|
| 1. | 8 μM MgSO₄ | 86.18° C. |
| 2. | 4 μM MgSO₄ + 4 μM Putrescine | 87.06° C. |
| 3. | 4 μM Putrescine | 87.62° C. |
| 4. | 4 μM MgSO₄ | 85.87° C. |
| 5. | 8 μM Putrescine | 89.01° C. |
| 6. | 4 μM MgSO₄ + 4 μM Spermidine | 86.90° C. |
| 7. | 4 μM Spermidine | 88.80° C. |
| 8. | No addition | 84.90° C. |

Since putrescine accelerated loop-mediated isothermal amplification (as measured by time to reach Ct) while spermidine did not (see previous example), the melting temperature data presented above suggests that the isothermal amplification accelerating effect seen with putrescine is not directly related to melting temperature.

### Example 3

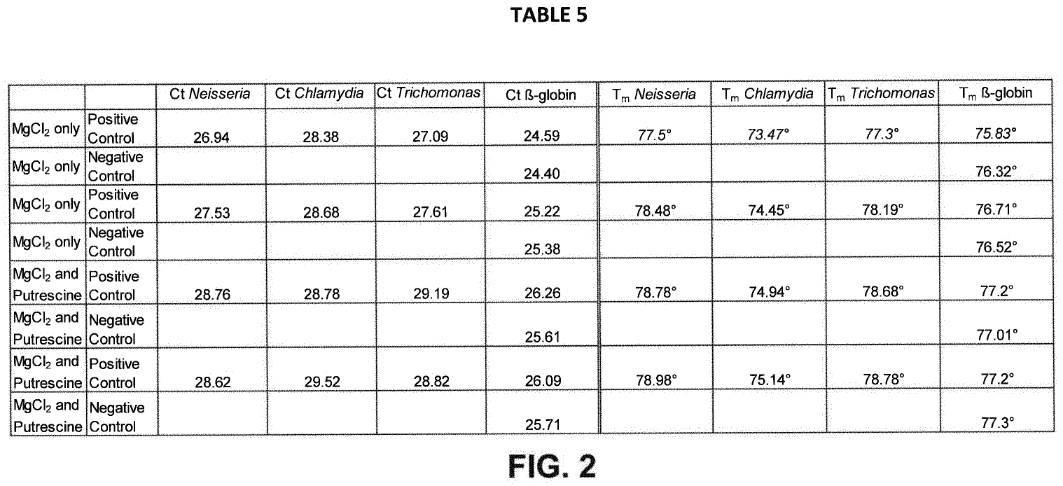

The effect of putrescine on PCR reactions was also examined. In these experiments, the effects of using MgCl₂ at 2.5 mM versus MgCl₂ at 1.25 mM with putrescine at 1.25 mM on PCR reactions were examined (see Table 5 appended as FIG. 2). The inclusion of putrescine did not decrease Ct in these reactions. Thus, contrary to the effects seen with LAMP, putrescine did not accelerate PCR and even appears to have impeded PCR to some extent in the experiments.

### Example 4

This experiment examined the effect of different additives on loop-mediated isothermal amplification at a lower magnesium concentration.

The buffer composition for the reaction is shown in Table 8 below.

TABLE 8

20 mM TrisHCl, pH 8.8
10 mM Potassium Chloride
15 mM Ammonium Sulfate
2.5 mM Magnesium Sulfate
0.1% Tween-20
960 mM Betaine
40 mM Guanidine-HCl
400 μM each of dATP, dCTP, dGTP and TTP
1/20,000 dilution of CyGreen
8 units Bst 2.0 Warmstart polymerase

The primers used are shown in Table 9 below:

TABLE 9

| | | |
|---|---|---|
| CTPlasLF372 | GCCGATGAGTTCGACATT (SEQ ID NO: 1) | 0.2 μM |
| CTPlasLR544 | TTTTAGAACTAAGTCTTCTGCTT (SEQ ID NO: 2) | 0.2 μM |
| CTPlasFL390 | CGCATATATTTTGGCCGCTAGAAAA | 1.6 μM |
| | CCACATACTTTCCCTATCACA (SEQ ID NO: 3) | |
| CTPlasRL543 | TAGGGGATCGATTGAAACTCTTTTT | 1.6 μM |
| | ACAATGCTCTTGCATATTACG (SEQ ID NO: 4) | |
| CTPlasLLF413 | GGCGATTTAAAAACCAAGGTC (SEQ ID NO: 25) | 0.4 μM |
| CTPlasLLR490 | GTAGAGTTTGGTTGGGGAGG (SEQ ID NO: 6) | 0.4 μM |
| CtOmp105FL | CACCAAGTGGTGCAAGGATCCGACGGAATTCTGTGGGAAG | 1.6 μM |
| | (SEQ ID NO: 7) | |
| CtOmp254RL | TATCAGCATGCGTATGGGTTACTA | 1.6 μM |
| | CCCATTTGGAATTCTTTATTCAC (SEQ ID NO: 8) | |
| CtOmp83FP | CTGCTGAACCAAGCCTTA (SEQ ID NO: 9) | 0.2 μM |
| CtOmp279RP | GCCTGTAGCAGTTGTAGG (SEQ ID NO: 10) | 0.2 μM |
| CtOmp370RP | AAGCGGCGTTTGTAAACAT (SEQ ID NO: 11) | 0.1 μM |
| CtOmp125LoopF | AAGGATCTCCGCCGAAAC (SEQ ID NO: 12) | 0.4 μM |
| CtOmp226LoopR | gtTTTCGACCGTGTTTTGCAAAC (SEQ ID NO: 13) | 0.4 μM |

4 mM amine or no amine was included as indicated. The reaction, run in duplicate, was incubated at 67° C. and fluorescence measurements were recorded every 30 seconds. The target was the Seracare Chlamydia/Neisseria Accurun 342 series 100 standards, heated to 95° C. for 3 minutes, then diluted 3 fold with water.

The results of this experiment are shown in Table 10 below:

TABLE 10

| | Ct | Ct |
|---|---|---|
| | positive | negative |
| | target | target |
| no addition | 14.89′ | — |
| Putrescine | 12.8′ | — |
| amino-butane | 18.58′ | — |
| 2,4,6-Triethyl-1,3,5- | 15.9′ | 40.01′ |
| benzenetrimethanamine | | |
| 1,3-diamino-2-propanol | 14.82′ | — |
| Imidazole | 16.52′ | — |

In this case, only Putrescine sped up the amplification reaction. The 2,4,6-Triethyl-1,3,5-benzenetrimethanamine caused false positives to appear. The monoamine amino-butane and imidazole at the magnesium concentration used appeared to delay the reaction.

### Example 5

The experimental setup is the same in this example as in Example 5 but different concentrations of Ammonium sulfate or Putrescine were examined.

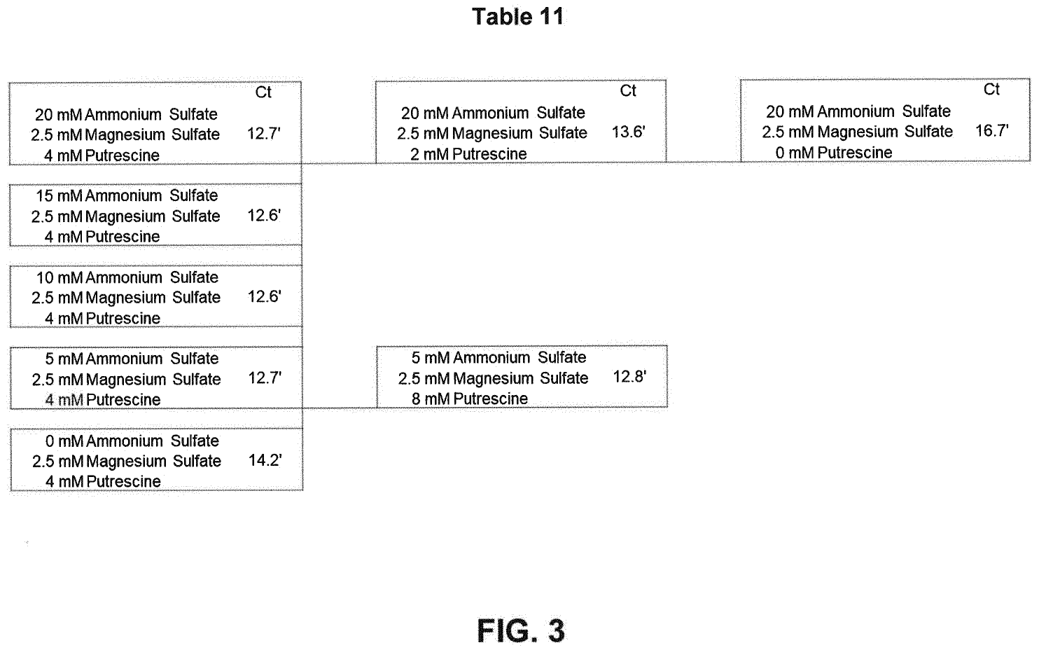

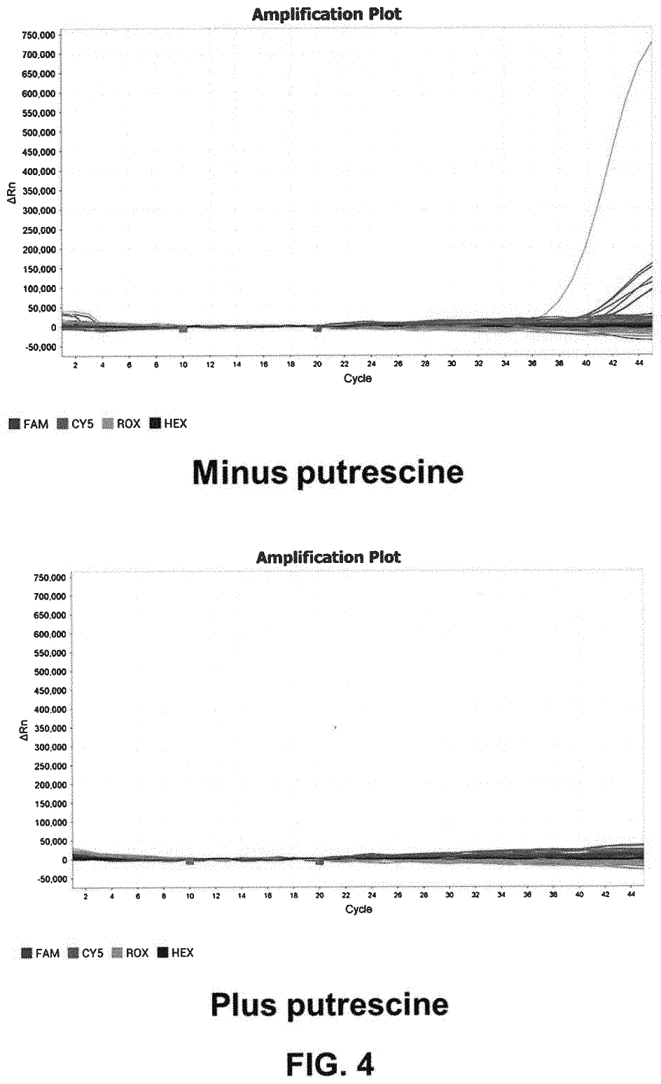

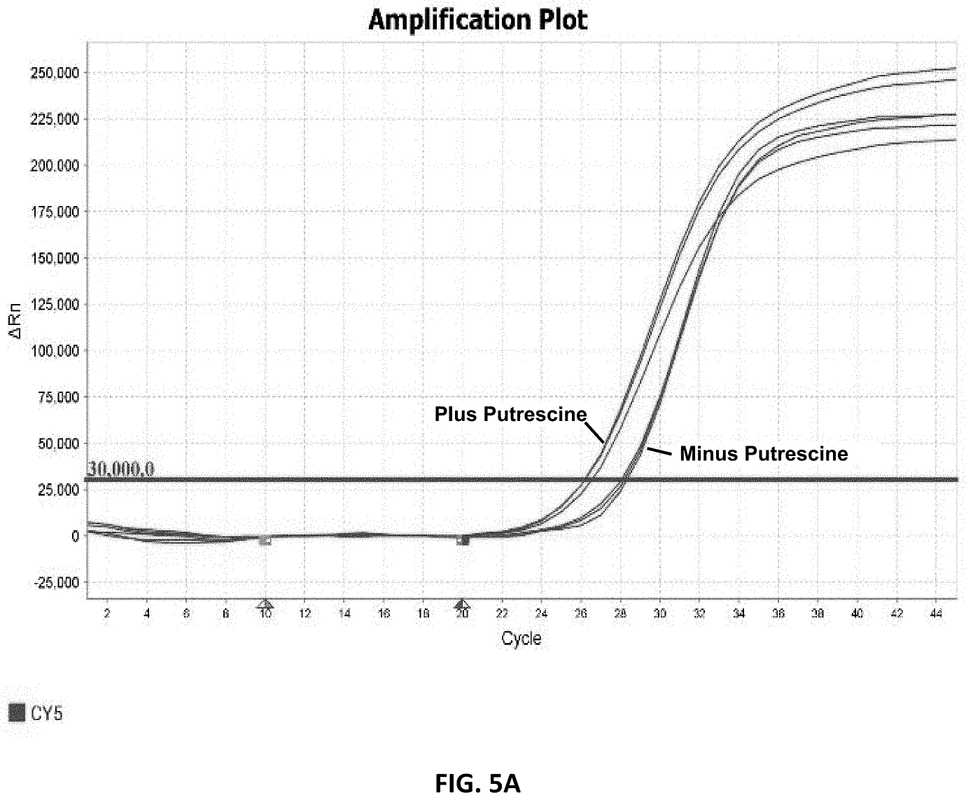

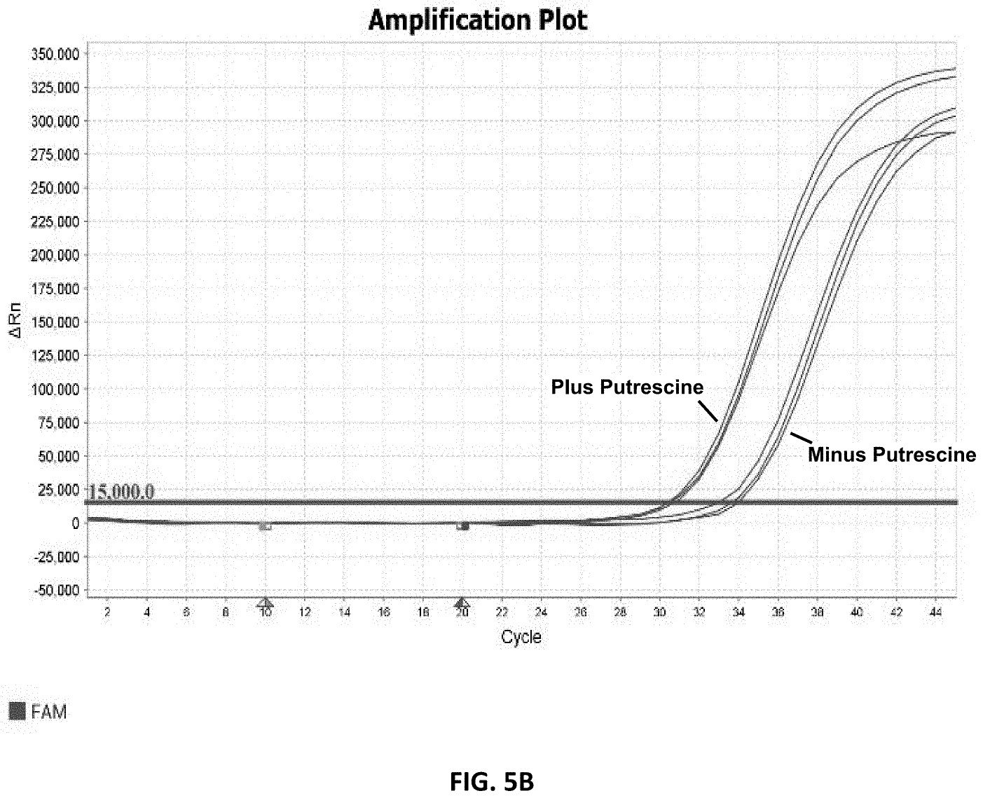

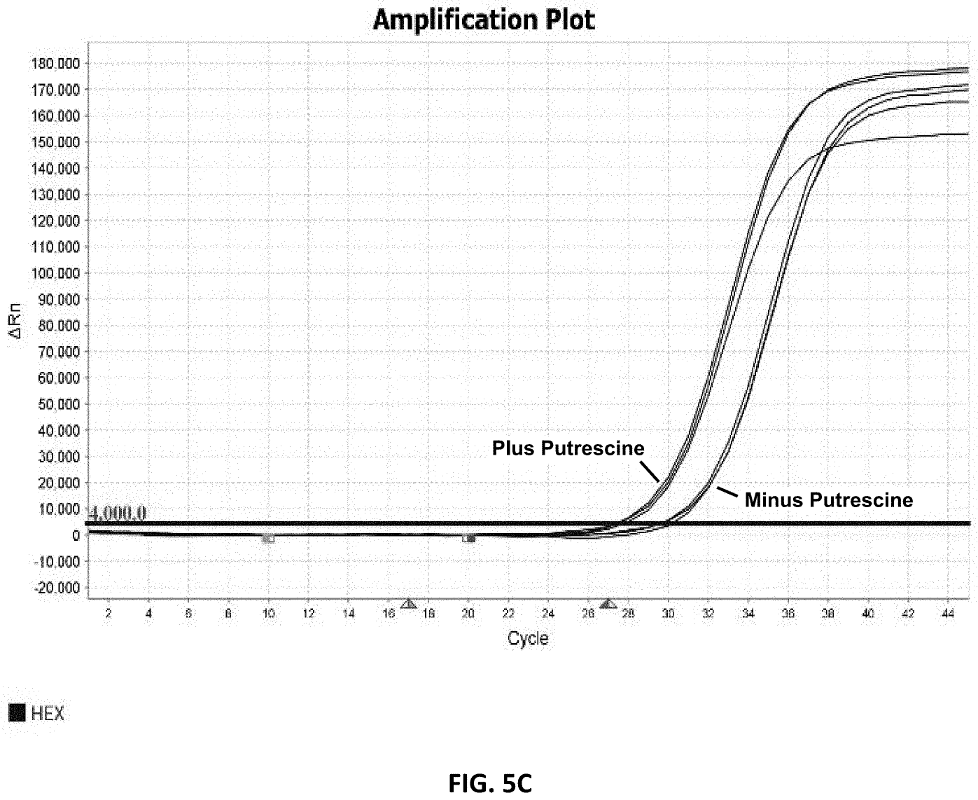

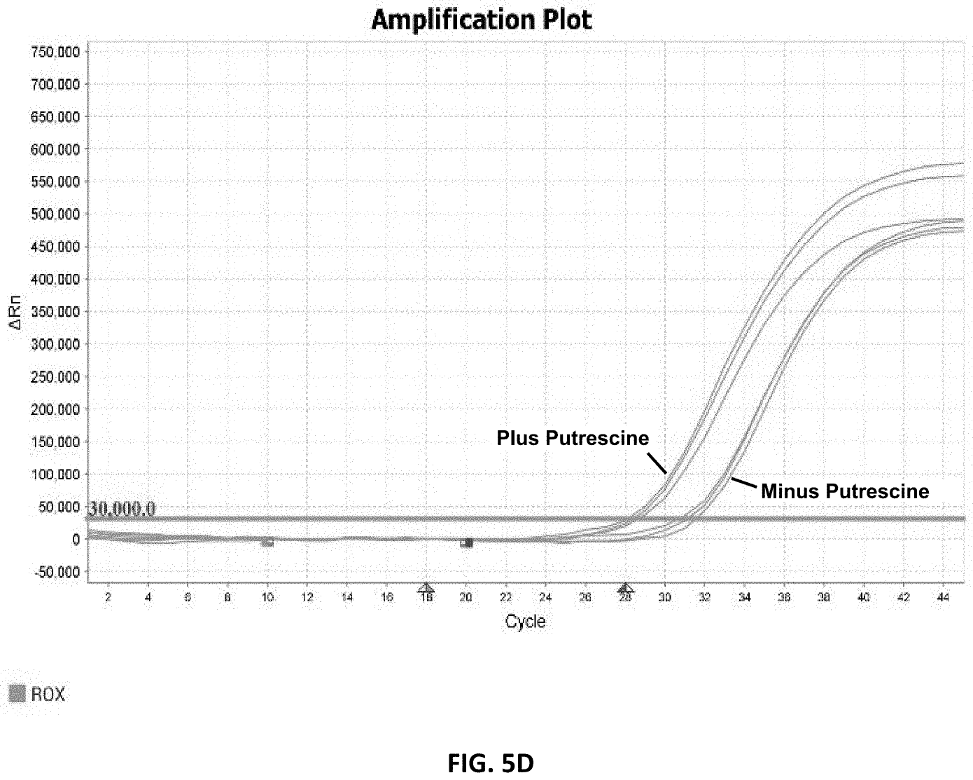

The result are shown in Table 11 provided in FIG. 3 appended hereto. The data is presented so that for each row only the Putrescine concentration is varied, and for each column only the ammonium sulfate concentration is varied. The results show that ammonium sulfate helps the reaction independently of Putrescine speeding up the amplification reaction given the other conditions under which the reaction occurs.

### Example 6

Prior to use in the reaction, the sample was incubated with 0.1% Tween20, 50 mM urea, 10 mM putrescine or dH₂O. The sample was either left at room temperature, or heated to 95° C. for 5 minutes. 10 μL of the sample was added to a 25 μL amplification in the mixture described in example 1, with 4 mM magnesium at 65° C. for 45 minutes.

The results of this experiment are shown in Table 12 below.

TABLE 12
Neisseria

| | |
|---|---|
| No additive heated | 15.9′ +/− 0.2′ |
| 50 mM urea heated | 16.1′ +/− 0.3′ |
| 0.1% Tween20 heated | 16.2′ +/− 0.1′ |
| No additive not heated | 18.6′ +/− 0.2′ |

Chlamydia

| | |
|---|---|
| No additive heated | 19.2′ +/− 0.2′ |
| 50 mM urea heated | 19.1′ +/− 0.3′ |
| 0.1% Tween20 heated | 19.3′ +/− 0.6′ |
| No additive not heated | 22.′ +/− 0.9′ |

Neisseria

| | |
|---|---|
| No additive not heated | 16.5′ +/− 0.2′ |
| 50 mM urea not heated | 16.7′ +/− 0.1′ |
| 0.1% Tween20 not heated | 16.8′ +/− 0.1′ |
| 10 mM putrescine | 17.′ +/− 0.2′ |
| 50 mM urea heated | 14.3′ +/− 0.4′ |
| No additive heated | 14.1′ |

Chlamydia

| | |
|---|---|
| No additive not heated | 18.5′ |
| 50 mM urea not heated | 19.1′ +/− 3.2′ |
| 0.1% Tween20 not heated | 16.8′ +/− 0.1′ |
| 10 mM putrescine | 19.5′ +/− 0.4′ |
| 50 mM urea heated | 16.4′ +/− 0.2′ |
| No additive heated | 16.9′ |

Neisseria/Chlamydia testing: dH₂O, urea, Tween 20, putrescine.

### Example 7

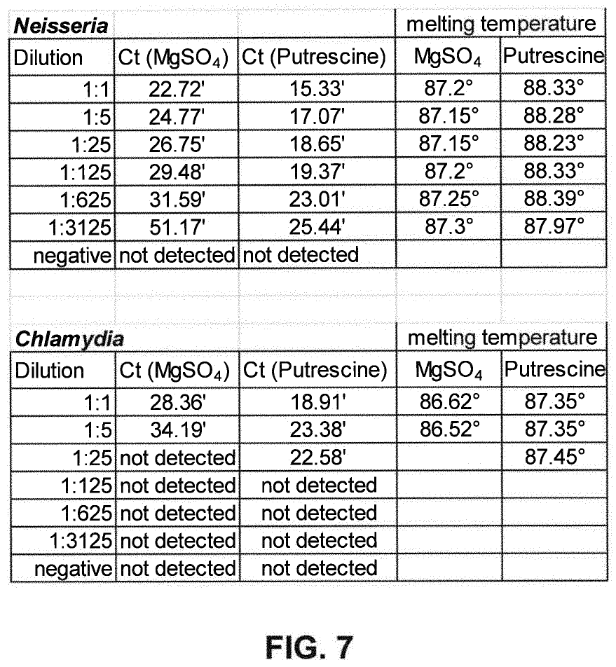

A titration was performed with Neisseria or Chlamydia as a target with or without putrescine. The target was the Seracare ACCURUN 342 Neisseria gonorrhea and Chlamydia trachomatis positive control. The target was heated to 95° C. for 5 minutes to release DNA from the organisms. 5 μL of target was used in a 25 μL reaction as described in example 1, with 4 mM magnesium with addition of 4 mM putrescine or an additional 4 mM magnesium. The results of this experiment are shown in FIG. 7.

### Example 8

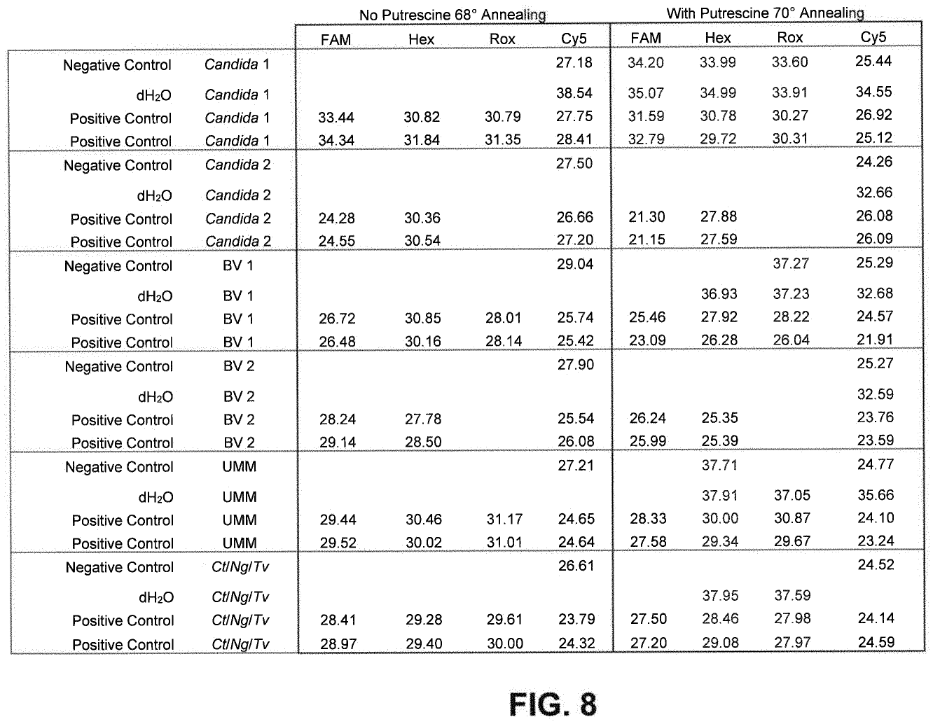

ENZO has developed the AmpiProbe method of nucleic acid amplification detection (U.S. Pat. No. 9,068,948) in which the forward primer has a fluorophore near the 3′ end, and the reverse primer has a quencher near the 3′ end. In Ampiprobe amplification, the 2 primers in the PCR type amplification are brought into close proximity, causing the signal to decay as more amplicon is produced. Using the AmpiProbe technology, ENZO has developed a Women's Health panel diagnostic that detects 5 species of Candida 5 species of Bacteria (BV), 3 species of Mycoplasma, and 3 sexually transmitted diseases (Ct/Ng/Tv). This panel was run as recommended, cycling from 95 C° for 10 seconds, 68° C. for 30 seconds, and reading the FAM, Hex, Rox and Cy5 channels after each cycle for 38 cycles. For template, the negative control that only contained the human beta-globin target, water, or the positive control that contained each target for the assay of interest was used. The same assay was run in the presence of 16 mM Putrescine, but extending at 70° C. instead of 68° C., to account for the difference in annealing temperature in the presence of the putrescine. As can be seen in FIG. 8, some of the targets appeared in earlier cycles in the presence of Putrescine, but also many false positives appeared in the reaction.

### Example 9

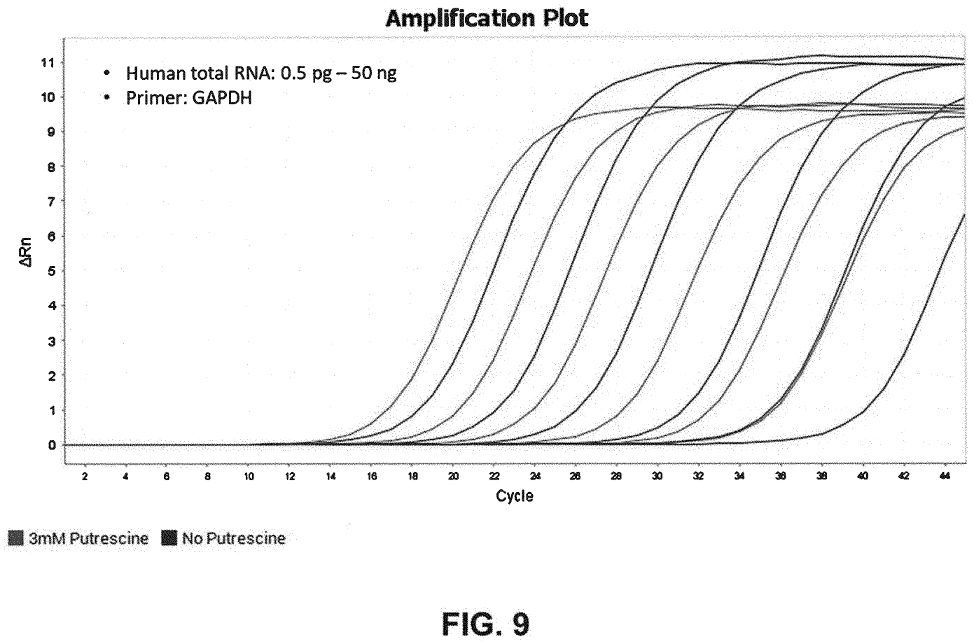

Putrescine was tested to see if it improves reverse transcriptase PCR reactions. The following primer set was used in a reverse transcriptase PCR targeting human GAPDH mRNA using the universal Green-ENZO RT-PCR kit: TCCAATATGATTCCACCCATG (SEQ ID NO:26) and GGAGGGATCTCGCTCCTG (SEQ ID NO:27). The cycling conditions were 50° C. for 30 minutes, 95° C. for 5 minutes, and 45 cycles of 94° C. for 15 seconds, 60° C. for 30 seconds, reading fluorescence at the end of each cycle in a QuantStudio 5 thermocycler. The target was a set of 10 fold serial dilutions of total human RNA from 50 nanograms down to 0.5 picograms. As can be seen in FIG. 9 if 3 mM putrescine was added in the reaction, the product was visualized earlier. FIG. 9 shows a serial dilution with putrescine added to Enzo Universal RT Mix.

### Example 10

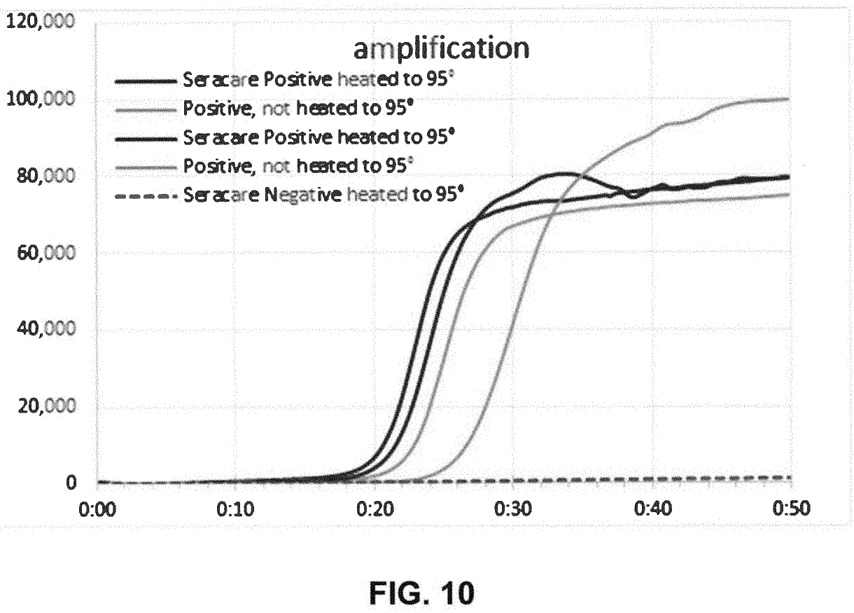

Neisseria gonorrhoeae was amplified as described in Example 1, at 67° C. in a 50 μl reaction in the presence of 22.2 μl of urine. Half of the positive samples (Seracare Chlamydia trichomonas/Neisseria gonorrhoeae controls) were heated to 95° C. for 3 minutes prior to amplification, and the other half was used without heating. FIG. 10 shows that the heated samples appear several minutes earlier than the unheated samples indicating that the heat treatment opens up the cells.

### Example 11

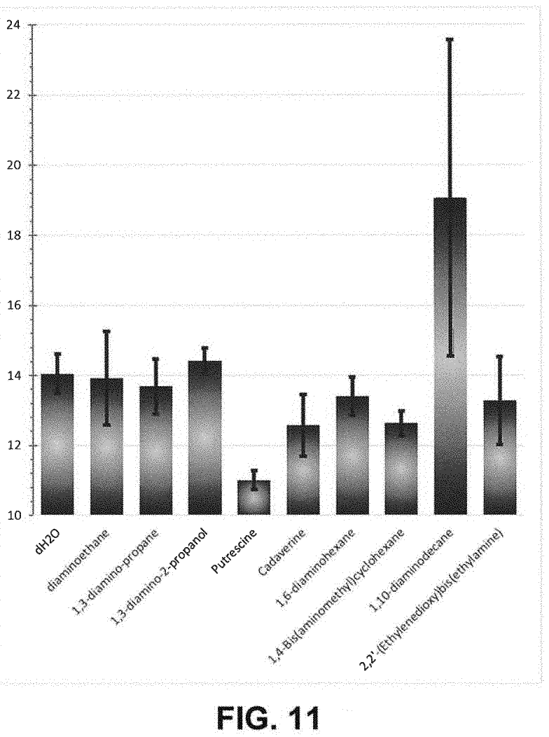

Using the protocol described in Example 1 for Chlamydia trachomatis, in a 25 si reaction at 65° C., different diamines were tested at 4 mM concentration for their effect on amplification rate in isothermal amplification. Diaminoethane, 1,3-diaminopropane, 1,3-diamino-2-propanol, putrescine, cadaverine, 1,6-diaminohexane, 1,4-Bis(aminomethylcyclohexane, 1,10-diaminodecane, and 2,2′-(Ethylenedideoxy)bis(ethylamine) were tested. The results of running the sample in triplicate are shown in FIG. 11. The compound 1,10-diaminodecane delayed amplification of Chlamydia in the isothermal amplification. Putrescine, Cadaverine, 1,6-diaminohexane, 1,4-Bis(aminomethyl)cyclohexane and 2,2′-(Ethylenedioxy)bis(ethylamine) decreased the time until the product was detected to varying degrees.

### Example 12

In a multiplexed PCR system, a diamine may allow a reduced primer concentration. For the normal amplification, the primers are 250 nM each. In this example, they were tested at 250 nM and 125 nM concentration. The primers used in the assay were:

| | |
|---|---|
| HSV1F-10 | TACCCCTGCCATCAACAC (SEQ ID NO: 28) |
| HSV1R-146 | GCGTGGGCATTTTCTGC (SEQ ID NO: 29) |
| HSV1-119P- | {FAM}ACTTCCGTGGCTTCTTGCTGC{BHQ1} (SEQ ID NO: 30) |
| HSV2F-227 | GCGACAATATCGTCTACGTC (SEQ ID NO: 31) |
| HSV2R-301 | GTGCTGCGTGTTGTAGAT (SEQ ID NO: 32) |
| HSV2-275P- | {Hex}AGCACCTGCCAGTAAGTCATCGG{BHQ1} (SEQ ID NO: 33) |
| VZVF-622 | CACAAACTCGCCGTCTTA (SEQ ID NO: 34) |
| VZVR-694 | CATGCGGAGAACAGAACA (SEQ ID NO: 35) |
| VarP-654 | {Calfluor610}CCTGAAGACGCACAACGCCTC{BHQ2} (SEQ ID NO: 36) |
| βglobF-1386 | TTGGCAAAGAATTCACCC (SEQ ID NO: 37) |
| βglobR-1479 | CGAGCTTAGTGATACTTGTG (SEQ ID NO: 38) |
| βglobP-1449 | {Quasar670}TTAGCCACACCAGCCACCACTT{BHQ2} (SEQ ID NO: 39) |

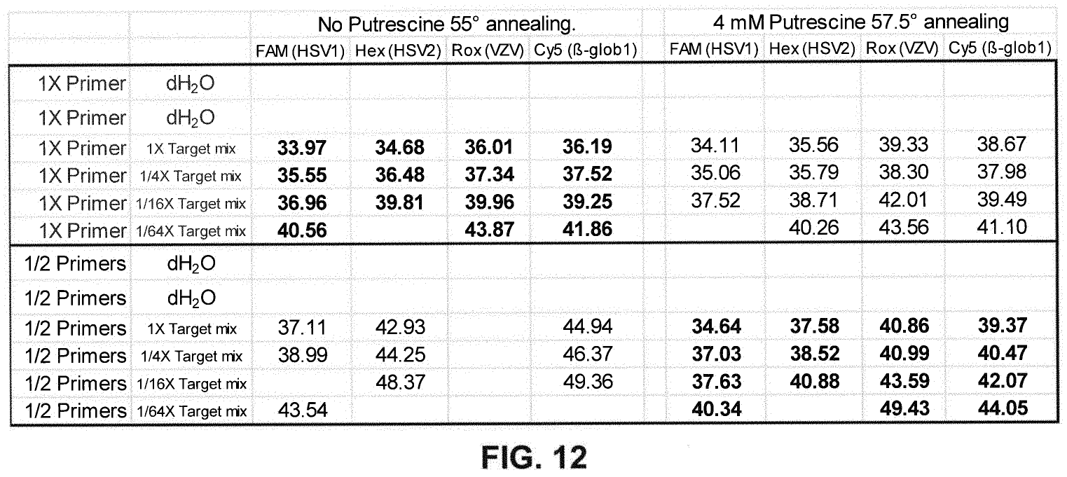

The assay used the ENZO Universal PCR Taq, with or without an additional 4 mM putrescine. The cycling conditions were 95° C. for 3 minutes to activate the enzyme, followed by 50 cycles of 95° C. for 5 seconds, 55° C. for 2 seconds, and 68° C. for 15 seconds. When putrescine was used, the annealing temperature was raised to 57.5° C. instead of 55° C., to account for the higher annealing temperature. The target in this experiment at 1× was 920 copies of a plasmid containing the given target. A series of 2 fold dilutions of the target was also tested. The Ct value for each target is given in FIG. 12.

This data shows that for some primer sets (HSV1) the presence of putrescine will allow a lower primer concentration. In other sets, the putrescine cannot return the Ct values of half primer concentration completely to the normal primer concentration value, but they are much better than without putrescine.

### Example 13

Candida krusei is a difficult to open organism. Methods to release DNA from the organism in order to detect it with isothermal amplification were tested. The Candida krusei (strain Castellani Berkhout) was from ATCC. The strain was incubated for 5 minutes in water, 800 mM urea, a mix of 700 mM urea and 23 mM ammonium sulfate, or 10 mM putrescine at either room temperature (23° C.) or 95° C.

After opening the cells, 5 μl of the cells mix was added into an isothermal amplification as described in example 1 using the primer set:

| | | |
|---|---|---|
| Ck-F305 | GCGAGTGTTGCGAGACAAC (SEQ ID NO: 40) | 0.2 μM |
| Ck-R521 | CAAGGGACTTGGACACCG (SEQ ID NO: 41) | 0.2 μM |
| Ck-LF344 | ACTGAGGCAATCCCTGTTGGTTAGGTAGGAATACCCGCTGAA | 1.6 μM |
| | (SEQ ID NO: 42) | |
| Ck-LR500 | TGAAGCGGCAAGAGCTCAGATTTCCACACAGACTCCAACCT | 1.6 μM |
| | (SEQ ID NO: 43) | |
| Ck-FL364 | CCTCCGCTTATTGATATGCTTAAG (SEQ ID NO: 44) | 0.4 μM |
| Ck-RL470 | GTGCTTTGCGGCACGAG (SEQ ID NO: 45) | 0.4 μM |

This was incubated at 65° C. for 45 minutes.

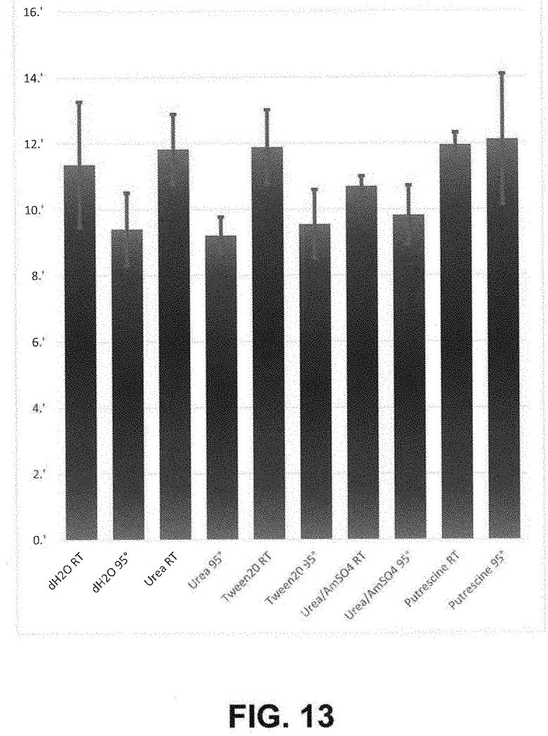

The resulting Ct values are shown in FIG. 13. The lower Ct values indicate that the DNA is more exposed or released from the cell.

### Example 14

The effect of different organic amines in replacing some of the magnesium in isothermal amplification was tested as described in example 1 with the primers targeting Neisseria gonorrhoeae or Chlamydia trichomonas from Seracare. The following were tested:

1. 8 mM magnesium sulfate
2. 4 mM magnesium sulfate with 4 mM putrescine
3. 4 mM magnesium sulfate with 4 mM cadaverine
4. 4 mM magnesium sulfate with 4 mM spermidine

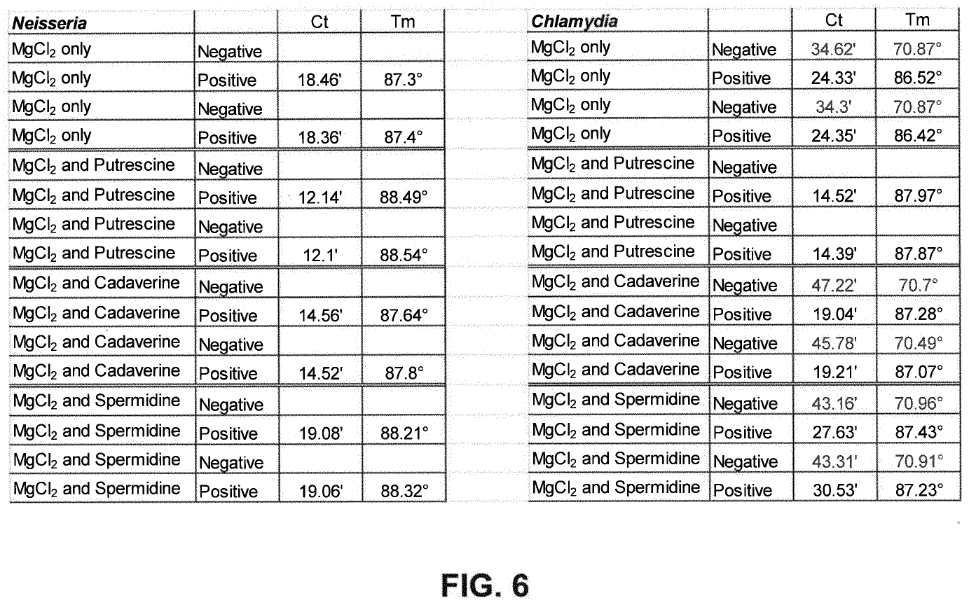

For both Neisseria and Chlamydia, putrescine and cadaverine made the reaction proceed faster, and spermidine slightly slows down the reaction. The melting curves indicate the each of these polyamines increased the melting temperature. The results are shown in FIG. 6.

### Example 15

Isothermal amplification of both Neisseria gonorrhoeae and Chlamydia trichomonas was performed as described in Example 4 using Seracare Chlamydia trichomonas/Neisseria gonorrhoeae controls diluted 8:10 with blood. To the sample was added 0.5% Tween 20, 0.5% Tween 20 plus 50 mM Putrescine, 1 M Urea, 1 M Urea plus 50 mM Putrescine, or no additive. These samples were heated to 95° C. for 5 minutes to release nucleic acid, then cooled to room temperature before isothermal amplification. All of the samples formed a large precipitate that prevented pipetting the sample for amplification. When the urea is added to the blood, such that the final concentration in the blood is 2 M urea prior to heating, there is not precipitate formed. 5 μl of this was added to a 25 μl reaction, and incubated for 45 minutes at 65° C. In this case, there was no precipitate formed, and amplification amplified normally.

Without limitation, the invention also provides the following enumerated embodiments:
1. A nucleic acid synthesis reaction mixture, comprising at least one organic diamine, an oligoamine, or other dication or combinations thereof.
2. The mixture of claim 1, wherein the at least one organic diamine is an aliphatic organic diamine.
3. The mixture of claim 2, wherein the at least one organic diamine is an alkyl diamine.
4. The mixture of claim 3, wherein the at least one organic diamine is a linear alkyl diamine.
5. The mixture of claim 4, wherein an amino group is disposed at each end of the linear alkyl diamine.
6. The mixture of claim 1, wherein the at least one organic diamine is selected from the group consisting of ethylenediamine, 1,3-diaminopropane, putrescine, cadaverine, 1,6-diaminohexane, or 2,2′-(Ethylenedixoy)bis(ethylamine) and combinations thereof.
7. The mixture of claim 6, wherein the at least one organic diamine is putrescine.
8. The mixture of claim 1, wherein the reaction mixture further comprises magnesium ion or magnesium salt.
9. The mixture of claim 1, wherein the at least one oligoamine is a metal ion in combination with a chelator.
10. The mixture of claim 1, wherein the at least one oligoamine is an organometallic compound.
11. A method for increasing the melting temperature of nucleic acids during nucleic acid synthesis, comprising providing at least one organic diamine, an oligoamine, or other dication to the nucleic acid synthesis.
12. The method of claim 11, wherein the at least one organic diamine is an aliphatic organic diamine.
13. The method of claim 12, wherein the at least one organic diamine is an alkyl diamine.
14. The method of claim 13, wherein the at least one organic diamine is a linear alkyl diamine.
15. The method of claim 14, wherein an amino group is disposed at each end of the linear alkyl diamine.
16. The method of claim 11, wherein the at least one organic diamine is selected from the group consisting of ethylenediamine, 1,3-diaminopropane, putrescine, cadaverine, 1,6-diaminohexane, or 2,2′-(Ethylenedixoy)bis(ethylamine) and combinations thereof.
17. The method of claim 16, wherein the at least one organic diamine is putrescine.
18. The method of claim 11, further comprising the addition of magnesium ion or magnesium salt to the nucleic acid synthesis.
19. The method of claim 11, wherein the at least one oligoamine is a metal ion in combination with a chelator.
20. The method of claim 11, wherein the at least one oligoamine is an organometallic compound.
21. A method for increasing the specific hybridization of nucleic acids during nucleic acid synthesis, comprising providing at least one organic diamine, an oligoamine, or other dication to the nucleic acid synthesis.
22. The method of claim 21, wherein the at least one organic diamine is an aliphatic organic diamine.
23. The method of claim 22, wherein the at least one organic diamine is an alkyl diamine.
24. The method of claim 23, wherein the at least one organic diamine is a linear alkyl diamine.
25. The method of claim 24, wherein an amino group is disposed at each end of the linear alkyl diamine.
26. The method of claim 21, wherein the at least one organic diamine is selected from the group consisting of ethylenediamine, 1,3-diaminopropane, putrescine, cadaverine, 1,6-diaminohexane, or 2,2′-(Ethylenedixoy)bis(ethylamine) and combinations thereof.
27. The method of claim 26, wherein the at least one organic diamine is putrescine.
28. The method of claim 21, further comprising the addition of magnesium ion or magnesium salt to the nucleic acid synthesis.
29. The method of claim 21, wherein the at least one oligoamine is a metal ion in combination with a chelator.
30. The method of claim 21, wherein the at least one oligoamine is an organometallic compound.
31. A method for increasing the rate of nucleic acid synthesis during a nucleic acid synthesis, comprising providing at least one organic diamine, an oligoamine, or other dication to the nucleic acid synthesis.
32. The method of claim 31, wherein the at least one organic diamine is an aliphatic organic diamine.
33. The method of claim 32, wherein the at least one organic diamine is an alkyl diamine.
34. The method of claim 33, wherein the at least one organic diamine is a linear alkyl diamine.
35. The method of claim 34, wherein an amino group is disposed at each end of the linear alkyl diamine.
36. The method of claim 31, wherein the at least one organic diamine is selected from the group consisting of ethylenediamine, 1,3-diaminopropane, putrescine, cadaverine, 1,6-diaminohexane, or 2,2′-(Ethylenedixoy)bis(ethylamine) and combinations thereof.
37. The method of claim 36, wherein the at least one organic diamine is putrescine.
38. The method of claim 31, further comprising the addition of magnesium ion or magnesium salt to the nucleic acid synthesis.
39. The method of claim 31, wherein the at least one oligoamine is a metal ion in combination with a chelator.
40. The method of claim 31, wherein the at least one oligoamine is an organometallic compound.
41. A method for improving the amplification of nucleic acids during a nucleic acid synthesis, comprising providing at least one organic diamine, an oligoamine, or other dication to the nucleic acid synthesis.
42. The method of claim 41, wherein the at least one organic diamine is an aliphatic organic diamine.
43. The method of claim 42, wherein the at least one organic diamine is an alkyl diamine.
44. The method of claim 43, wherein the at least one organic diamine is a linear alkyl diamine.
45. The method of claim 44, wherein an amino group is disposed at each end of the linear alkyl diamine.
46. The method of claim 41, wherein the at least one organic diamine is selected from the group consisting of ethylenediamine, 1,3-diaminopropane, putrescine, cadaverine, 1,6-diaminohexane, or 2,2′-(Ethylenedixoy)bis(ethylamine) and combinations thereof.
47. The method of claim 46, wherein the at least one organic diamine is putrescine.
48. The method of claim 41, further comprising the addition of magnesium ion or magnesium salt to the nucleic acid synthesis.
49. The method of claim 41, wherein the at least one oligoamine is a metal ion in combination with a chelator.
50. The method of claim 41, wherein the at least one oligoamine is an organometallic compound.
51. A method for reducing the background signal in a nucleic acid synthesis, comprising providing at least one organic diamine, an oligoamine, or other dication to the nucleic acid synthesis.
52. The method of claim 51, wherein the at least one organic diamine is an aliphatic organic diamine.
53. The method of claim 52, wherein the at least one organic diamine is an alkyl diamine.
54. The method of claim 53, wherein the at least one organic diamine is a linear alkyl diamine.
55. The method of claim 54, wherein an amino group is disposed at each end of the linear alkyl diamine.
56. The method of claim 51, wherein the at least one organic diamine is selected from the group consisting of ethylenediamine, 1,3-diaminopropane, putrescine, cadaverine, 1,6-diaminohexane, or 2,2′-(Ethylenedixoy)bis(ethylamine) and combinations thereof.
57. The method of claim 56, wherein the at least one organic diamine is putrescine.
58. The method of claim 51, further comprising the addition of magnesium ion or magnesium salt to the nucleic acid synthesis.
59. The method of claim 51, wherein the at least one oligoamine is a metal ion in combination with a chelator.
60. The method of claim 51, wherein the at least one oligoamine is an organometallic compound.
61. A sample processing method for nucleic acid detection that improves or eliminates at least one of sample preparation, lysing, separating, washing, or elution, comprising heating the sample in the presence of either urine or urea, and introducing a portion or all of said heated sample directly into a nucleic acid detection system.
62. The method of claim 61, wherein the detection is not inhibited.
63. The method of claim 61, wherein the amount of the sample used is 10% of the volume of the nucleic acid detection reaction.
64. The method of claim 61, wherein the amount of the sample used is 20% of the volume of the nucleic acid detection reaction.
65. The method of claim 61, wherein the amount of the sample used is 30% of the volume of the nucleic acid detection reaction.
66. The method of claim 61, wherein the amount of the sample used is 40% of the volume of the nucleic acid detection reaction.
67. The method of claim 61, wherein the amount of the sample used is 50% of the volume of the nucleic acid detection reaction.
68. The method of claim 61, wherein the concentration of urea prior to heating is from about 1 M to about 4 M.
69. The method of claim 68, wherein the concentration of urea prior to heating is from about 1.5 M to about 3 M.
70. The method of claim 61, wherein the separating is by binding to a matrix.
71. The method of claim 61, wherein the separating is by precipitation.
72. A process for identifying a compound that both increases the melting temperature of nucleic acids and enhances specific hybridization during nucleic acid synthesis, comprising
   contacting a compound of interest with a nucleic acid having a known melting temperature during nucleic acid synthesis and a known temperature at which hybridization occurs during nucleic acid synthesis and measuring the melting temperature of the nucleic acid after contact with the compound of interest and measuring the temperature at which hybridization occurs after contact with the compound of interest during nucleic acid synthesis, and selecting compounds that both increase the melting temperature during nucleic acid synthesis and the temperature at which hybridization occurs for said known nucleic acid.
73. A process for identifying a compound that enhances reverse transcriptase synthesis, comprising
   contacting a compound of interest with a nucleic acid and measuring whether the reverse transcriptase synthesis is more efficient than in the absence of said compound, and selecting compounds that improve the efficiency of reverse transcriptase synthesis.

It should also be understood that wherever in the present application the term comprising or including (or a term of similar scope) is recited in connection with the description of any embodiment or part thereof, a corresponding embodiment or part thereof reciting instead the term consisting essentially of or the term consisting of (or a term of similar scope) is also disclosed.

Any and all publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s). Moreover, features described in connection with one embodiment of the invention may be used in conjunction with other embodiments, even if not explicitly exemplified in combination within.

1. A nucleic acid synthesis reaction mixture, comprising at least one organic diamine, an oligoamine, or other dication or combinations thereof.

2. The mixture of claim 1, wherein the at least one organic diamine is an aliphatic organic diamine.

3. The mixture of claim 2, wherein the at least one organic diamine is an alkyl diamine.

4. The mixture of claim 3, wherein the at least one organic diamine is a linear alkyl diamine.

5. The mixture of claim 4, wherein an amino group is disposed at each end of the linear alkyl diamine.

6. The mixture of claim 1, wherein the at least one organic diamine is selected from the group consisting of ethylenediamine, 1,3-diaminopropane, putrescine, cadaverine, 1,6-diaminohexane, or 2,2′-(Ethylenedixoy)bis(ethylamine) and combinations thereof.

7. The mixture of claim 6, wherein the at least one organic diamine is putrescine.

8. The mixture of claim 1, wherein the reaction mixture further comprises magnesium ion or magnesium salt.

9. The mixture of claim 1, wherein the at least one oligoamine is a metal ion in combination with a chelator.

10. The mixture of claim 1, wherein the at least one oligoamine is an organometallic compound.

11. A method for increasing the melting temperature of nucleic acids during nucleic acid synthesis, comprising
providing at least one organic diamine, an oligoamine, or other dication to the nucleic acid synthesis.

12. The method of claim 11, wherein the at least one organic diamine is an aliphatic organic diamine.

13. The method of claim 12, wherein the at least one organic diamine is an alkyl diamine.

14. The method of claim 13, wherein the at least one organic diamine is a linear alkyl diamine.

15. The method of claim 14, wherein an amino group is disposed at each end of the linear alkyl diamine.

16. The method of claim 11, wherein the at least one organic diamine is selected from the group consisting of ethylenediamine, 1,3-diaminopropane, putrescine, cadaverine, 1,6-diaminohexane, or 2,2′-(Ethylenedixoy)bis(ethylamine) and combinations thereof.

17. The method of claim 16, wherein the at least one organic diamine is putrescine.

18. The method of claim 11, further comprising the addition of magnesium ion or magnesium salt to the nucleic acid synthesis.

19. The method of claim 11, wherein the at least one oligoamine is a metal ion in combination with a chelator.

20. The method of claim 11, wherein the at least one oligoamine is an organometallic compound.

21. A method for increasing the specific hybridization of nucleic acids during nucleic acid synthesis, comprising
providing at least one organic diamine, an oligoamine, or other dication to the nucleic acid synthesis.

22. The method of claim 21, wherein the at least one organic diamine is an aliphatic organic diamine.

23. The method of claim 22, wherein the at least one organic diamine is an alkyl diamine.

24. The method of claim 23, wherein the at least one organic diamine is a linear alkyl diamine.

25. The method of claim 24, wherein an amino group is disposed at each end of the linear alkyl diamine.

26. The method of claim 21, wherein the at least one organic diamine is selected from the group consisting of ethylenediamine, 1,3-diaminopropane, putrescine, cadaverine, 1,6-diaminohexane, or 2,2′-(Ethylenedixoy)bis(ethylamine) and combinations thereof.

27. The method of claim 26, wherein the at least one organic diamine is putrescine.

28. The method of claim 21, further comprising the addition of magnesium ion or magnesium salt to the nucleic acid synthesis.

29. The method of claim 21, wherein the at least one oligoamine is a metal ion in combination with a chelator.

30. The method of claim 21, wherein the at least one oligoamine is an organometallic compound.

31. A method for increasing the rate of nucleic acid synthesis during a nucleic acid synthesis, comprising
providing at least one organic diamine, an oligoamine, or other dication to the nucleic acid synthesis.

32. The method of claim 31, wherein the at least one organic diamine is an aliphatic organic diamine.

33. The method of claim 32, wherein the at least one organic diamine is an alkyl diamine.

34. The method of claim 33, wherein the at least one organic diamine is a linear alkyl diamine.

35. The method of claim 34, wherein an amino group is disposed at each end of the linear alkyl diamine.

36. The method of claim 31, wherein the at least one organic diamine is selected from the group consisting of ethylenediamine, 1,3-diaminopropane, putrescine, cadaverine, 1,6-diaminohexane, or 2,2′-(Ethylenedixoy)bis(ethylamine) and combinations thereof.

37. The method of claim 36, wherein the at least one organic diamine is putrescine.

38. The method of claim 31, further comprising the addition of magnesium ion or magnesium salt to the nucleic acid synthesis.

39. The method of claim 31, wherein the at least one oligoamine is a metal ion in combination with a chelator.

40. The method of claim 31, wherein the at least one oligoamine is an organometallic compound.

41. A method for improving the amplification of nucleic acids during a nucleic acid synthesis, comprising
providing at least one organic diamine, an oligoamine, or other dication to the nucleic acid synthesis.

42. The method of claim 41, wherein the at least one organic diamine is an aliphatic organic diamine.

43. The method of claim 42, wherein the at least one organic diamine is an alkyl diamine.

44. The method of claim 43, wherein the at least one organic diamine is a linear alkyl diamine.

45. The method of claim 44, wherein an amino group is disposed at each end of the linear alkyl diamine.

46. The method of claim 41, wherein the at least one organic diamine is selected from the group consisting of ethylenediamine, 1,3-diaminopropane, putrescine, cadaverine, 1,6-diaminohexane, or 2,2′-(Ethylenedixoy)bis(ethylamine) and combinations thereof.

47. The method of claim 46, wherein the at least one organic diamine is putrescine.

48. The method of claim 41, further comprising the addition of magnesium ion or magnesium salt to the nucleic acid synthesis.

49. The method of claim 41, wherein the at least one oligoamine is a metal ion in combination with a chelator.

50. The method of claim 41, wherein the at least one oligoamine is an organometallic compound.

51. A method for reducing the background signal in a nucleic acid synthesis, comprising
providing at least one organic diamine, an oligoamine, or other dication to the nucleic acid synthesis.

52. The method of claim 51, wherein the at least one organic diamine is an aliphatic organic diamine.

53. The method of claim 52, wherein the at least one organic diamine is an alkyl diamine.

54. The method of claim 53, wherein the at least one organic diamine is a linear alkyl diamine.

55. The method of claim 54, wherein an amino group is disposed at each end of the linear alkyl diamine.

56. The method of claim 51, wherein the at least one organic diamine is selected from the group consisting of ethylenediamine, 1,3-diaminopropane, putrescine, cadaverine, 1,6-diaminohexane, or 2,2′-(Ethylenedixoy)bis(ethylamine) and combinations thereof.

57. The method of claim 56, wherein the at least one organic diamine is putrescine.

58. The method of claim 51, further comprising the addition of magnesium ion or magnesium salt to the nucleic acid synthesis.

59. The method of claim 51, wherein the at least one oligoamine is a metal ion in combination with a chelator.

60. The method of claim 51, wherein the at least one oligoamine is an organometallic compound.

61. A sample processing method for nucleic acid detection that improves or eliminates at least one of sample preparation, lysing, separating, washing, or elution, comprising
heating the sample in the presence of either urine or urea, and
introducing a portion or all of said heated sample directly into a nucleic acid detection system.

62. The method of claim 61, wherein the detection is not inhibited.

63. The method of claim 61, wherein the amount of the sample used is 10% of the volume of the nucleic acid detection reaction.

64. The method of claim 61, wherein the amount of the sample used is 20% of the volume of the nucleic acid detection reaction.

65. The method of claim 61, wherein the amount of the sample used is 30% of the volume of the nucleic acid detection reaction.

66. The method of claim 61, wherein the amount of the sample used is 40% of the volume of the nucleic acid detection reaction.

67. The method of claim 61, wherein the amount of the sample used is 50% of the volume of the nucleic acid detection reaction.

68. The method of claim 61, wherein the concentration of urea prior to heating is from about 1 M to about 4 M.

69. The method of claim 68, wherein the concentration of urea prior to heating is from about 1.5 M to about 3 M.

70. The method of claim 61, wherein the separating is by binding to a matrix.

71. The method of claim 61, wherein the separating is by precipitation.

72. A process for identifying a compound that both increases the melting temperature of nucleic acids and enhances specific hybridization during nucleic acid synthesis, comprising
contacting a compound of interest with a nucleic acid having a known melting temperature during nucleic acid synthesis and a known temperature at which hybridization occurs during nucleic acid synthesis and measuring the melting temperature of the nucleic acid after contact with the compound of interest and measuring the temperature at which hybridization occurs after contact with the compound of interest during nucleic acid synthesis, and selecting compounds that both increase the melting temperature during nucleic acid synthesis and the temperature at which hybridization occurs for said known nucleic acid.

73. A process for identifying a compound that enhances reverse transcriptase synthesis, comprising
contacting a compound of interest with a nucleic acid and measuring whether the reverse transcriptase synthesis is more efficient than in the absence of said compound, and selecting compounds that improve the efficiency of reverse transcriptase synthesis.
It should also be understood that wherever in the present application the term comprising or including (or a term of similar scope) is recited in connection with the description of any embodiment or part thereof, a corresponding embodiment or part thereof reciting instead the term consisting essentially of or the term consisting of (or a term of similar scope) is also disclosed.
Any and all publications, patents, patent applications and other documents cited in this application are hereby incorporated by reference in their entireties for all purposes to the same extent as if each individual publication, patent, patent application or other document were individually indicated to be incorporated by reference for all purposes.
While various specific embodiments have been illustrated and described, it will be appreciated that various changes can be made without departing from the spirit and scope of the invention(s). Moreover, features described in connection with one embodiment of the invention may be used in conjunction with other embodiments, even if not explicitly exemplified in combination within.
